(19) United States
(12) Patent Application Publication
(10) Pub. No.: US 2014/0106446 A1
(43) Pub. Date: Apr. 17, 2014

(54) METHODS AND COMPOSITIONS FOR LONG TERM HEMATOPOIETIC REPOPULATION

(71) Applicant: University of Louisville Research Foundation, Inc., (unknown)

(72) Inventors: Janina Ratajczak, Louisville, KY (US); Ewa K. Zuba-Surma, Cracow (PL); Mariusz Z. Ratajczak, Louisville, KY (US)

(73) Assignee: University of Louisville Research Foundation, Inc.

(21) Appl. No.: 14/136,436

(22) Filed: Dec. 20, 2013

Related U.S. Application Data
(63) Continuation of application No. 13/129,359, filed on Jan. 17, 2012, now abandoned.
(60) Provisional application No. 61/199,356, filed on Nov. 14, 2008.

(51) Int. Cl. A61K 35/48

(52) U.S. Cl. CPC A61K 35/51
USPC 435/325

(57) ABSTRACT

Methods for isolating a CD133⁺/CD45^{neg}/GlyA^{neg} subpopulation of umbilical cord blood cells are disclosed. In some embodiments, the methods include providing an initial population of umbilical cord blood cells; contacting the initial population of cells with a first antibody that is specific for CD133, a second antibody that is specific for CD45, and a third antibody that is specific for Glycophorin A (GlyA) under conditions sufficient to allow binding of each antibody to its target, if present, on each cell of the initial population of cells; and isolating a subpopulation of cells that are CD133⁺, CD45^{neg}, and GlyA^{neg}. Also provided are isolated populations of CD133⁺/GlyA^{neg}/CD45^{neg} stem cells isolated from cord blood, methods for repopulating cell types in subjects, methods for bone marrow transplantation, methods for inducing hematopoietic competency in CD133⁺/GlyA^{neg}/CD45^{neg} stem cells, and cell culture systems that include CD133⁺/GlyA^{neg}/CD45^{neg} stem cells.

### CROSS REFERENCE TO RELATED APPLICATIONS

This application is a continuation of U.S. patent application Ser. No. 13/129,359, filed May 13, 2012, which itself is a National Stage application of PCT International Patent Application Serial No. PCT/US2009/064614, filed Nov. 16, 2009, which itself claimed the benefit of U.S. Provisional Patent Application Ser. No. 61/199,356, filed Nov. 14, 2008. The disclosure of each of these applications is incorporated herein by reference in its entirety.

### GRANT STATEMENT

This invention was made with government support under grants R01 CA106281-01 and R01 DK074720 awarded by the National Institutes of Health of the United States of America. The government has certain rights in the invention.

### TECHNICAL FIELD

The presently disclosed subject matter relates in some embodiments to methods for repopulating a cell type in a subject. In some embodiments the presently disclosed subject matter relates to administering to a subject in need thereof a composition comprising a plurality of isolated cord blood-derived CD133⁺/GlyA^{neg}/CD45^{neg} stem cells in an amount and via a route sufficient to allow at least a fraction of the cord blood-derived for repopulating a cell type in a subject to engraft a target site in the subject and differentiate therein, whereby a cell type is repopulated in the subject.

### BACKGROUND

Progress in hematological transplantology has increased the demand for hematopoietic stem cells (HSCs) isolated from histocompatible donors. It is well known that suitable bone marrow (BM) donors are often in short supply. Unfortunately, cord blood (CB) contains a much lower absolute number of HSCs than BM, making the CB less preferred for treatment use in adult patients. In addition, it is currently very difficult to reliably expand long term repopulating (LT)-HSCs isolated from BM- and CB-HSCs, exacerbating the need for a new supply of LT-HSCs.

Thus, it has been postulated that embryonic stem cell-derived HSCs might have a number of advantages over HSCs isolated from conventional sources such as BM and CB. This, however, has proven difficult to employ since strategies to differentiate embryonic stem cells (ESCs) along the hematopoietic lineage are difficult to employ and optimize. Moreover, human ESCs are the subject of various restrictions that limit their availability and usefulness, even for experimental studies.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

The presently disclosed subject matter provides methods for isolating a CD133⁺/CD45^{neg}/GlyA^{neg} subpopulation of umbilical cord blood cells. In some embodiments, the methods comprise (a) providing an initial population of umbilical cord blood cells; (b) contacting the initial population of cells with a first antibody that is specific for CD133, a second antibody that is specific for CD45, and a third antibody that is specific for Glycophorin A (GlyA) under conditions sufficient to allow binding of each antibody to its target, if present, on each cell of the initial population of cells; and (c) isolating a subpopulation of cells that are CD133⁺, CD45^{neg}, and GlyA^{neg}. In some embodiments, the contacting step comprises simultaneously or iteratively contacting the umbilical cord blood cells with a plurality of antibodies that specifically bind to CD133, GlyA, and CD45. In some embodiments, the methods further comprise isolating ALDH^{high} cells from the CD133⁺/GlyA^{neg}/CD45^{neg} cells, ALDH^{low} cells from the CD133⁺/GlyA^{neg}/CD45^{neg} cells, or both ALDH^{high} cells and ALDH^{low} cells separately from the CD133⁺/GlyA^{neg}/CD45^{neg} cells.

The presently disclosed subject matter also provides isolated populations of stem cells that comprise substantially purified CD133⁺/GlyA^{neg}/CD45^{neg} cells isolated from cord blood (CB). In some embodiments, the CD133⁺/GlyA^{neg}/CD45^{neg} cells are ALDH^{high} cells. In some embodiments, the CD133⁺/GlyA^{neg}/CD45^{neg} cells are ALDH^{low} cells.

The presently disclosed subject matter also provides compositions comprising the presently disclosed isolated populations of stem cells. In some embodiments, the compositions further comprise one or more pharmaceutically acceptable carriers and/or excipients. In some embodiments, the pharmaceutically acceptable carriers and/or excipients are pharmaceutically acceptable for use in a human.

The presently disclosed subject matter also provides methods for repopulating a cell type in a subject. In some embodiments, the methods comprise administering to the subject a composition comprising a plurality of isolated CD133⁺/GlyA^{neg}/CD45^{neg} stem cells in a pharmaceutically acceptable carrier in an amount and via a route sufficient to allow at least a fraction of the CD133⁺/GlyA^{neg}/CD45^{neg} stem cells to engraft a target site and differentiate therein, whereby a cell type is repopulated in the subject. In some embodiments, the cell type is a hematopoietic cell. In some embodiments, the target site comprises the bone marrow. In some embodiments, the subject is a mammal. In some embodiments, the mammal is a human. In some embodiments, the plurality of isolated CD133⁺/GlyA^{neg}/CD45^{neg} stem cells comprises CD133⁺/GlyA^{neg}/CD45^{neg} stem cells isolated from cord blood. In some embodiments, the pharmaceutically acceptable carrier is pharmaceutically acceptable for use in a human.

The presently disclosed subject matter also provides methods for bone marrow transplantation. In some embodiments, the methods comprise administering to a subject with at least partially absent bone marrow a pharmaceutical preparation comprising an effective amount of CD133⁺/GlyA^{neg}/CD45^{neg} stem cells isolated from cord blood, wherein the effective amount comprises an amount of isolated CD133⁺/GlyA^{neg}/CD45^{neg} stem cells sufficient to engraft in the bone marrow of the subject. In some embodiments, the subject with at least partially absent bone marrow has undergone a pre-treatment to at least partially reduce the bone marrow in the subject. In some embodiments, the pre-treatment comprises a myeloreductive or a myeloablative treatment. In some embodiments, the pre-treatment comprises administering to the subject an immunotherapy, a chemotherapy, a radiation therapy, or a combination thereof. In some embodiments, the radiation therapy comprises total body irradiation. In some embodiments, the administering comprises intravenous administration of the pharmaceutical preparation. In some embodiments, the CD133⁺/GlyA^{neg}/CD45^{neg} stem cells are CD133⁺/GlyA^{neg}/CD45^{neg}/ALDH^{high} stem cells. In some embodiments, the methods further comprise co-culturing the CD133⁺/GlyA^{neg}/CD45^{neg} stem cells in the presence of an OP9 cell feeder layer for at least 5 days prior to the administering step.

The presently disclosed subject matter also provides methods for inducing hematopoietic competency in a CD133⁺/GlyA^{neg}/CD45^{neg} stem cell. In some embodiments, the methods comprise (a) providing a CD133⁺/GlyA^{neg}/CD45^{neg} stem cell; and (b) co-culturing the CD133⁺/GlyA^{neg}/CD45^{neg} stem cell in the presence of an OP9 feeder layer for a time sufficient to induce hematopoietic competency in the CD133⁺/GlyA^{neg}/CD45^{neg} stem cell. In some embodiments, the CD133⁺/GlyA^{neg}/CD45^{neg} stem cells are bone marrow-derived CD133⁺/GlyA^{neg}/CD45^{neg} stem cells, cord blood-derived CD133⁺/GlyA^{neg}/CD45^{neg} stem cells, or a combination thereof. In some embodiments, the CD133⁺/GlyA^{neg}/CD45^{neg} stem cells are CD133⁺/GlyA^{neg}/CD45^{neg}/ALDH^{low} stem cells. In some embodiments, the CD133⁺/GlyA^{neg}/CD45^{neg} stem cells are CD133⁺/GlyA^{neg}/CD45^{neg}/ALDH^{high} stem cells. In some embodiments, the hematopoietic competency comprises an ability to engraft bone marrow in a subject when the CD133⁺/GlyA^{neg}/CD45^{neg} stem cell is administered to the subject. In some embodiments, the hematopoietic competency comprises an ability to provide long term engraftment of the bone marrow in the subject. In some embodiments, the time sufficient to induce hematopoietic competency comprises at least 5 days of co-culturing. In some embodiments, the presently disclosed methods further comprise isolating the CD133⁺/GlyA^{neg}/CD45^{neg} stem cell from human cord blood.

The presently disclosed subject matter also provides cell culture systems comprising CD133⁺/GlyA^{neg}/CD45^{neg} stem cells. In some embodiments, the cell culture systems also comprise an OP9 cell feeder layer. In some embodiments, the CD133⁺/GlyA^{neg}/CD45^{neg} stem cells are human cord blood CD133⁺/GlyA^{neg}/CD45^{neg} stem cells, human bone marrow CD133⁺/GlyA^{neg}/CD45^{neg} stem cells, or a combination thereof. In some embodiments, the CD133⁺/GlyA^{neg}/CD45^{neg} stem cells are CD133⁺/GlyA^{neg}/CD45^{neg}/ALDH^{high} stem cells.

Thus, it is an object of the presently disclosed subject matter to provide methods for isolating a CD133⁺/CD45^{neg}/GlyA^{neg} subpopulation of umbilical cord blood cells.

An object of the presently disclosed subject matter having been stated hereinabove, and which is achieved in whole or in part by the presently disclosed subject matter, other objects will become evident as the description proceeds when taken in connection with the accompanying Figures as best described herein below.

### BRIEF DESCRIPTION OF THE FIGURES

FIGS. 1A and 1B are a schematic approach to isolating ALDH^{low} and ALDH^{high} CB-VSELs by a combined strategy that employs Magnetic Cell Sorting (MACS) followed by Fluorescence Activated Cell Sorting (FACS) separations, and a representative gating strategy for FACS isolation of subpopulations of CB-VSELs based on ALDH activity, respectively.

FIG. 2 is a schematic diagram of a technique for in vitro expansion of ALDH^{low} and ALDH^{high} subpopulation of CB-VSELs. Freshly isolated subpopulations of cells were cultured in methylcellulose clonogenic assays (tope panel) or expanded for 5 days over an OP9 cell feeder layer (bottom panel) and subsequently tested for a number of clonogenic progenitors in methylcellulose cloning assays.

FIG. 3 is a bar graph showing the total number of hematopoietic colonies (CFUs) obtained in clonogenic culture from ALDH^{low} and ALDH^{high} subpopulations of CB-VSELs. The numbers of colonies were calculated per 1×10³ sorted cells of each population. The values presented are Mean±SEM; *: p<0.05; N=5.

FIG. 4 is a set of two photomicrographs of “Cobble-stone” areas formed by ALDH^{low} and ALDH^{high} subpopulations of CD133⁺/GlyA^{neg}/CD45^{neg} CB-VSELs in co-culture with OP9 cells. Both photomicrographs are brightfield images. The bars in the bottom left corner of each photomicrograph indicate 10 μm. The spindle-like shaped OP9 cells are shown to form a feeder layers in the culture plates.

FIG. 5 is a set of two micrographs of colonies obtained in clonogenic methylcellulose assays from ALDH^{low} and ALDH^{high} subpopulations of CD133⁺/GlyA^{neg}/CD45^{neg} CB-VSELs expanded over OP9 feeder cells. Both photos present brightfield images. The bars in the bottom left corner of each photomicrograph indicate 10 μm.

FIGS. 6A and 6B are a bar graph and a photomicrograph, respectively, showing CD45 expression of cells harvested from clonogenic cultures initiated by ALDH^{low} and ALDH^{high} CB-VSELs.

FIG. 6A shows the expression of CD45 antigen on cells harvested from clonogenic cultures initiated by ALDH^{low} and ALDH^{high} CB-VSELs analyzed by flow cytometry. FIG. 6B shows representative images of cells obtained from ALDH^{low} CB-VSELs in clonogenic cultures that were subsequently re-plated into single-cell culture, stained for CD45 (TRITC), and analyzed by epifluorescence microscopy. Comparison of the left and right panels shows a CD45^{neg} cell indicated by the black arrow in the left panel and several CD45⁺ cells indicated by the white arrow in the right panel. The scale bar shown in the left panel indicates 10 μm, and the scale is the same for both panels.

FIG. 7 is a series of representative epifluorescence images of colonies derived from CD133⁺/GlyA^{neg}/CD45^{neg}/ALDH^{low} and CD133⁺/GlyA^{neg}/CD45^{neg}/ALDH^{high} CB-VSELs stained for Glycophorin A (upper panels) or CD45 (lower panels). All images are shown in the same magnification, and the scale bars indicate 10 μm.

FIGS. 8A and 8B are bar graphs showing expression of genes related to pluripotent stage and hematopoietic commitment in ALDH^{low} and ALDH^{high} fractions of CB-VSELs.

FIG. 8A shows expression of genes related to pluripotent stage and hematopoietic commitment in ALDH^{low} and ALDH^{high} fractions of CB-VSELs directly after isolation, and FIG. 8B shows expression of genes related to pluripotent stage and hematopoietic commitment in ALDH^{low} and ALDH^{high} fractions of CB-VSELs after co-culture over OP9 cells followed by clonogenic culture. The fold-difference numbers presented on the y-axes represent average values (Mean±SEM). *: p<0.05 vs. total nucleated cells (TNCs).

FIG. 9A is a bar graph showing absolute numbers of CB-VSELs and HSCs that can be isolated from fraction of TNCs (isolated after lysis of RBCs) and mononuclear cells (MNCs; after Ficoll-Paque separation). Data are expressed per 1 ml of processed CB.

FIG. 9B is a bar graph showing size and nuclear to cytoplasmic (N/C ratio) of CB-VSELs as compared to HSCs. The values present Mean±SEM. *: p<0.05; N=5.

FIGS. 10A and 1B are bar graphs that show the hematopoietic potential of CB-derived CD45^{neg}/CD133⁺/ALDH^{high} and CD45^{neg}/CD133⁺/ALDH^{low} VSELs tested in vivo after transplantation into lethally-irradiated NOD/SCID mice assayed 4-6 weeks after transplantation.

FIG. 10A is a bar graph showing the contributions of CB-derived CD45^{neg}/CD133⁺/ALDH^{high} and CD45^{neg}/CD133⁺/ALDH^{low} VSELs to hematopoietic cells in the peripheral blood (PB), spleen (SP), and bone marrow (BM) of transplanted mice. The levels of human hematopoietic CD45⁺ derived from the subpopulations of CB-derived VSELs in murine PB, BM, and SP were comparable between the two transplanted CB-VSELs fractions: 7.1±2.9% (PB), 23.2±0.2% (SP), and 25.2±1.0% (BM).

FIG. 10B is a bar graph showing the extent of reconstitution of hematopoietic lineages in the peripheral blood of NOD/SCID mice. CD3 is a T cell marker, CD19 is a B cell marker (although it is also expressed on expressed on follicular dendritic cells), CD66b is a granulocyte marker, and GlyA is a marker for the erythroid lineage.

FIG. 11 is a schematic diagram of a potential mechanism for developmental deposition of epiblast-derived embryonic stem cells in adult tissues. The presence of VSELs in the fetal liver, BM and other tissues could be explained by the developmental deposition of CXCR4⁺ epiblast-derived VSELs that follow an SDF-1 gradient. Fetal liver can function as an important crossroad in the migratory route of these cells.

FIG. 12 shows the results of flow cytometric analyses of the contents of various populations in FL showing a gating strategy for analysis of VSELs content (Sca-1⁺/Lin^{neg}/CD45^{neg} cells).

FIGS. 13A and 13B are bar graphs showing expression of markers of pluripotent stem cells and tissue-committed stem cells, and the content of VSELs and the VSEL-DS-forming capacity of fetal liver cells at various stages of development, respectively. Sca-1⁺Lin^{neg}CD45^{neg} FL-derived cells express several markers of PSCs and grow spheres in co-cultures with C2C12 myoblasts. The values represent average numbers obtained from three independent experiments (Mean±SEM). Fetal livers from 15-20 fetuses were combined in each experiment

FIG. 13A is a bar graph showing analysis of mRNA expression for several genes characterizing pluripotent stem cells (PSCs) and tissue-committed stem cells (TCSCs) in sorted fractions of Sca-1⁺/Lin^{neg}/CD45^{neg} FL-derived cells when compared with fetal liver cells mononuclear cells. Analysis was performed in different time points after fertilization.

FIG. 13B is a bar graph showing the correlation of percent content of Sca-1⁺/Lin^{neg}/CD45^{neg} FL-derived cells and absolute number of VSEL-derived spheres (VSEL-DS) cultured in vitro from sorted Sca-1⁺/Lin^{neg}/CD45^{neg} in relation to total FL cells.

FIG. 14 is a series of IMAGESTREAM® System (ISS) analyses of content and morphology of FL-derived VSELs. FL-derived cells were stained antibodies specific for Sca-1 (conjugated to FITC), Lin markers (each conjugated to PE), and CD45 (conjugated to PE-Cy5™), fixed with paraformaldehyde solution (2%), permeabilized with TRITON™ X (0.01%) and analyzed by ISS. FIG. 14 shows the identification of Sca-1⁺/Lin^{neg}/CD45^{neg} cells based on their size and antigenic profile in FL at 15.5 dpc. The upper left plot shows all of the analyzed objects according to their morphological parameters including nuclear area and aspect ratio on brightfield. The aspect ratio is calculated based on brightfield cellular image as the ratio of cellular minor axis (width) to major axis (height) (round, non-elongated cells possess aspect ratio close to 1.0, while the elongated cells or clumps have lower aspect ratio). Round, single cells with DNA content were included in region R1 and further analyzed for the expression of CD45. CD45^{neg} cells from region R2 were analyzed for Lin markers expression and Lin^{neg}/CD45^{neg} cells were enclosed in region R3. Cells from this region were subsequently visualized based on Sca-1 expression and Sca-1⁺/Lin^{neg}/CD45^{neg} cells were included into region R4.

FIG. 15 is two graphs that summarize changes in absolute numbers at days 12.5, 15.5, and 17.5 dpc in fetal liver of Sca-1⁺/Lin^{neg}/CD45^{neg} cells (black squares) and Oct-4⁺/Sca-1⁺/Lin^{neg}/CD45^{neg} VSELs (gray circles; left graph) as well as Sca-1⁺/Lin^{neg}/CD45⁺ HSCs (right graph).

### DETAILED DESCRIPTION

Primitive LT-HSCs can maintain long term hematopoiesis when engrafted into appropriate recipients. While the existence of these cells has been demonstrated experimentally, the phenotype and hence the specific isolation of such cells remains controversial.

Mounting evidence indicates that BM contains a population of pluripotent (P)SCs that can give rise to LT-HSCs (Kucia et al. (2006) Leukemia 20:857-869). Recently, during analysis of murine BM, a homogenous population of rare (−0.01% of BM mononuclear cells (MNCs)) and very small (about 2-4 μm) Sca-1⁺/lin^{neg}/CD45^{neg} cells that express PSC markers such as SSEA-1, Oct-4, Nanog, and Rex-1 and that highly express Rif-1 telomerase protein were discovered (Kucia et al. (2006) Leukemia 20:857-869). Direct electron microscopic analysis revealed that these cells displayed several features typical for primary epiblast-derived ESCs such as a large nuclei surrounded by a narrow rim of cytoplasm and open-type chromatin (euchromatin). In co-cultures with a C2C12 murine sarcoma-supportive feeder layer, these cells grew spheres composed of immature CXCR4⁺/SSEA-1⁺/Oct-4⁺ cells having large nuclei that contain euchromatin. When plated into cultures promoting tissue differentiation, these cells showed pluripotency and expanded into cells from all three germ-cell layers. Based on this, these cells were referred to as very small, embryonic-like (VSEL) SCs (see also PCT International Patent Application Publication Nos. WO 2007/067280 and 2009/059032).

Disclosed herein are studies that focus on hematopoietic differentiation of these cells. It is believed that VSELs could be the most primitive population of PSCs in BM and that they are able to differentiate along the hematopoietic lineage and give rise to LT-HSCs. As set forth herein, VSELs freshly isolated from the BM do not posses immediate hematopoietic activity; they neither grow hematopoietic colonies nor radioprotect lethally-irradiated recipients. However, if CD45^{neg} VSELs are plated over a supportive OP9 cell line, they gave rise to colonies of CD45⁺/CD41⁺/Gr1⁺/Ter119⁺ cells. The phenotype of these cells resembled those of the earliest hematopoietic cells derived in vitro from established embryonic cell lines. This hematopoietic differentiation of VSELs was accompanied by upregulation of mRNA for several genes regulating hematopoiesis (e.g., PU-1, c-myb, LMO2, and Ikaros). More importantly, the CD45+/CD41^{neg}/Gr-1^{neg}/Ter119^{neg} cells expanded from VSELs isolated from GFP⁺ mice when transplanted into wild-type (WT) animals. These protected the WTs from lethal irradiation and differentiated in vivo into all major hematopoietic lineages (e.g., Gr-1⁺, B220⁺, and CD3⁺ cells). This hematopoietic activity was maintained after transplantation into secondary recipients. Based on this, it appears that VSELs are PSCs that can give rise to LT-HSCs, and further that CD45⁺ cells might derive from a CD45^{neg} population.

### I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. For example, the phrase “a cell” refers to one or more cells, including, but not limited to a plurality of the same cell type or a plurality of different cell types. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent present in the pharmaceutical composition. It is noted, however, that carriers, excipients, and other inactive agents can and likely would be present in the pharmaceutical composition.

With respect to the terms “comprising”, “consisting essentially of”, and “consisting of”, where one of these three terms is used herein, the presently disclosed and claimed subject matter can include the use of either of the other two terms. For example, the presently disclosed subject matter relates in some embodiments to compositions that comprise CD133⁺/GlyA^{neg}/CD45^{neg} cells. It is understood that the presently disclosed subject matter thus also encompasses compositions that in some embodiments consist essentially of CD133⁺/GlyA^{neg}/CD45^{neg} cells, as well as compositions that in some embodiments consist of CD133⁺/GlyA^{neg}/CD45^{neg} cells. Similarly, it is also understood that in some embodiments the methods of the presently disclosed subject matter comprise the steps the steps that are disclosed herein and/or that are recited in the claims, in some embodiments the methods of the presently disclosed subject matter consist essentially of the steps that are disclosed herein and/or that are recited in the claims, and in some embodiments the methods of the presently disclosed subject matter consist of the steps that are disclosed herein and/or that are recited in the claim.

As used herein, the phrase “long term” when used in the context of bone marrow transplantation refers to a period of time in which the donor cell or a progeny cell derived therefrom remains viable and functional in the donor. Bone marrow transplantation is considered to result in long term engraftment when hematopoietic cells derived from the donor cells are present in the recipient for in some embodiments at least 3 months, in some embodiments 6 months, in some embodiments 9 months, in some embodiments 12 months, and in some embodiments for longer than 12 months after administration.

### II. Methods for Isolating Subpopulations of Umbilical Cord Blood Cells

In some embodiments, the presently disclosed subject matter provides methods for isolating a CD133⁺/CD45^{neg}/GlyA^{neg} subpopulation of umbilical cord blood (CB) cells. In some embodiments, the methods comprise (a) providing an initial population of umbilical cord blood cells; (b) contacting the initial population of cells with a first ligand (e.g., an antibody) that is specific for CD133, a second ligand (e.g., an antibody) that is specific for CD45, and a third ligand (e.g., an antibody) that is specific for Glycophorin A (GlyA) under conditions sufficient to allow binding of each antibody to its target, if present, on each cell of the initial population of cells; and (c) isolating a subpopulation of cells that are CD133⁺, CD45^{neg}, and GlyA^{neg}.

Thus, in some embodiments the presently disclosed subject matter provides methods of isolating a subpopulation of CD45^{neg} stem cells from a population of CB cells. In some embodiments, the method comprises (a) providing a population of CB cells suspected of comprising CD45^{neg} stem cells; (b) contacting the population of CB cells with a first antibody that is specific for CD45, a second antibody that is specific for CD133, and a under conditions sufficient to allow binding of each antibody to its target, if present, on each cell of the population of cells; (c) selecting a first subpopulation of CB cells that are CD133⁺ and are also CD45^{neg}; (d) contacting the first subpopulation of CB cells with one or more antibodies that are specific for one or more cell surface markers selected from the group including but not limited to CD45R/B220, Gr-1, TCRaβ, TCRγδ, CD11b, and Ter-119 under conditions sufficient to allow binding of each antibody to its target, if present, on each cell of the population of CB cells; (e) removing from the first subpopulation of CB cells those cells that bind to at least one of the antibodies of step (d); and (f) collecting a second subpopulation of CB cells that are either CD133⁺/CD45^{neg}/GlyA^{neg}, whereby a subpopulation of CD45^{neg} stem cells is isolated.

As used herein, the term “CD45” refers to a tyrosine phosphatase, also known as the leukocyte common antigen (LCA), and having the gene symbol PTPRC. This gene corresponds to GENBANK® Accession Nos. NP_{—}002829 (human), NP_{—}035340 (mouse), NP_{—}612516 (rat), XP_{—}002829 (dog), XP_{—}599431 (cow) and AAR16420 (pig). The amino acid sequences of additional CD45 homologs are also present in the GENBANK® database, including those from several fish species and several non-human primates.

As used herein, the term “CD34” refers to a cell surface marker found on certain hematopoietic and non-hematopoietic stem cells, and having the gene symbol CD34. The GENBANK® database discloses amino acid and nucleic acid sequences of CD34 from humans (e.g., AAB25223), mice (NP_{—}598415), rats (XP_{—}223083), cats (NP_{—}001009318), pigs (MP_{—}999251), cows (NP_{—}776434), and others.

In mice, some stem cells also express the stem cell antigen Sca-1 (GENBANK® Accession No. NP 034868), also referred to as Lymphocyte antigen Ly-6A.2.

As used herein, the term “CD133” refers to a cell surface marker found on certain in hematopoietic stem cells, endothelial progenitor cells, glioblastomas, neuronal and glial stem cells, and some other cell types. It is also referred to as Prominin 1 (PROM1). The GENBANK® database discloses nucleic acid and amino acid sequences of CD133 from humans (e.g., NM_{—}006017 and NP_{—}006008), mice (NM_{—}008935 and NP_{—}032961), rats (NM_{—}021751 and NP_{—}068519), and others.

As used herein, the term “GlyA” refers to glycophorin A, a cell surface molecule present on red blood cells. The GENBANK® database discloses nucleic acid and amino acid sequences of GlyA from humans (e.g., NM_{—}002099 and NP_{—}002090), mice (NM_{—}010369 and NP_{—}034499), and others.

Thus, the subpopulation of CD45^{neg} stem cells represents a subpopulation of CD45^{neg} cells that are present in the population of cells prior to the separating step. In some embodiments, the subpopulation of CD45^{neg} stem cells are from a human, and are CD34⁺/lin^{neg}/CD45^{neg}. In some embodiments, the subpopulation of CD45^{neg} stem cells are from a mouse, and are Sca-1⁺/lin^{neg}/CD45^{neg}.

The isolation of the disclosed subpopulations can be performed using any methodology that can separate cells based on expression or lack of expression of the one or more of the CD45, CD133, GlyA, CXCR4, CD34, AC133, Sca-1, CD45R/B220, Gr-1, TCRaβ, TCRγδ, CD11b, and Ter-119 markers including, but not limited to fluorescence-activated cell sorting (FACS).

As used herein, lin^{neg} refers to a cell that does not express any of the following markers: CD45R/B220, Gr-1, TCRaβ, TCRγδ, CD11b, and Ter-119. These markers are found on cells of the B cell lineage from early Pro-B to mature B cells (CD45R/B220); cells of the myeloid lineage such as monocytes during development in the bone marrow, bone marrow granulocytes, and peripheral neutrophils (Gr-1); thymocytes, peripheral T cells, and intestinal intraepithelial lymphocytes (TCRaβ and TCRγδ); myeloid cells, NK cells, some activated lymphocytes, macrophages, granulocytes, B1 cells, and a subset of dendritic cells (CD11b); and mature erythrocytes and erythroid precursor cells (Ter-119).

The separation step can be performed in a stepwise manner as a series of steps or concurrently. For example, the presence or absence of each marker can be assessed individually, producing two subpopulations at each step based on whether the individual marker is present. Thereafter, the subpopulation of interest can be selected and further divided based on the presence or absence of the next marker.

Alternatively, the subpopulation can be generated by separating out only those cells that have a particular marker profile, wherein the phrase “marker profile” refers to a summary of the presence or absence of two or more markers. For example, a mixed population of cells can contain both CD133⁺ and CD34^{neg} cells. Similarly, the same mixed population of cells can contain both CD45⁺ and CD45^{neg} cells. Thus, certain of these cells will be CD133⁺/CD45⁺, others will be CD133⁺/CD45^{neg}, others will be CD133^{neg}/CD45⁺, and others will be CD133^{neg}/CD45^{neg}. Each of these individual combinations of markers represents a different marker profile. As additional markers are added, the profiles can become more complex and correspond to a smaller and smaller percentage of the original mixed population of cells. In some embodiments, the cells of the presently disclosed subject matter have a marker profile of CD133⁺/CD45^{neg}/GlyA^{neg}.

In some embodiments of the presently disclosed subject matter, antibodies specific for markers expressed by a cell type of interest (e.g., polypeptides expressed on the surface of a CD133⁺/CD45^{neg}/GlyA^{neg} cell are employed for isolation and/or purification of subpopulations of BM cells that have marker profiles of interest. It is understood that based on the marker profile of interest, the antibodies can be used to positively or negatively select fractions of a population, which in some embodiments are then further fractionated.

In some embodiments, a plurality of antibodies, antibody derivatives, and/or antibody fragments with different specificities is employed. In some embodiments, each antibody, or fragment or derivative thereof, is specific for a marker selected from the group including but not limited to CD133, CD45, GlyA, Ly-6A/E (Sca-1), CD34, CXCR4, AC133, CD45, CD45R, B220, Gr-1, TCRαβ, TCRγδ, CD11b, Ter-119, c-met, LIF-R, SSEA-1, Oct-4, Rev-1, and Nanog. In some embodiments, cells that express one or more genes selected from the group including but not limited to SSEA-1, Oct-4, Rev-1, and Nanog are isolated and/or purified.

The presently disclosed subject matter relates to a population of cells that in some embodiments express the following antigens: CXCR4, AC133, CD34, SSEA-1 (mouse) or SSEA-4 (human), fetal alkaline phosphatase (AP), c-met, and the LIF-Receptor (LIF-R). In some embodiments, the cells of the presently disclosed subject matter do not express the following antigens: CD45, lineage markers (i.e., the cells are lin^{neg}), GlyA, HLA-DR, MHC class I, CD90, CD29, and CD105. Thus, in some embodiments the cells of the presently disclosed subject matter can be characterized as follows: CXCR4⁺/CD133⁺/CD34⁺/SSEA-1⁺ (mouse) or SSEA-4⁺ (human)/AP⁺/c-met⁺/LIF-R⁺/CD45^{neg}/lin^{neg}/HLA-DR^{neg}/MHC class I^{neg}/GlyA^{neg}/CD90^{neg}/CD29^{neg}/CD105^{neg}.

It is understood that in order to isolate a subpopulation of cells with the marker profile desired (e.g., CD133⁺/CD45^{neg}/GlyA^{neg}), the ligands that are used to separate cells based on expression of the relevant markers (e.g., antibodies) can be employed simultaneously or iteratively, in any combination that is convenient. For example, antibodies that bind to CD133, CD45, and GlyA can be employed simultaneously, in any desired combinations, or single in any order to separate the desired subpopulations.

In some embodiments, each antibody, or fragment or derivative thereof, comprises a detectable label. Different antibodies, or fragments or derivatives thereof, which bind to different markers can comprise different detectable labels or can employ the same detectable label.

A variety of detectable labels are known to the skilled artisan, as are methods for conjugating the detectable labels to biomolecules such as antibodies and fragments and/or derivatives thereof. As used herein, the phrase “detectable label” refers to any moiety that can be added to an antibody, or a fragment or derivative thereof, that allows for the detection of the antibody. Representative detectable moieties include, but are not limited to, covalently attached chromophores, fluorescent moieties, enzymes, antigens, groups with specific reactivity, chemiluminescent moieties, and electrochemically detectable moieties, etc. In some embodiments, the antibodies are biotinylated. In some embodiments, the biotinylated antibodies are detected using a secondary antibody that comprises an avidin or streptavidin group and is also conjugated to a fluorescent label including, but not limited to Cy3, Cy5, and Cy7. In some embodiments, the antibody, fragment, or derivative thereof is directly labeled with a fluorescent label such as Cy3, Cy5, or Cy7. In some embodiments, the antibodies comprise biotin-conjugated rat anti-mouse Ly-6A/E (Sca-1; clone E13-161.7), streptavidin-PE-Cy5 conjugate, anti-CD45-APCCy7 (clone 30-F11), anti-CD45R/B220-PE (clone RA3-6B2), anti-Gr-1-PE (clone RB6-8C5), anti-TCRαβ PE (clone H57-597), anti-TCRγδ PE (clone GL3), anti-CD11b PE (clone M1/70) and anti-Ter-119 PE (clone TER-119). In some embodiments, the antibody, fragment, or derivative thereof is directly labeled with a fluorescent label and cells that bind to the antibody are separated by fluorescence-activated cell sorting. Additional detection strategies are known to the skilled artisan.

While FACS scanning is a convenient method for purifying subpopulations of cells, it is understood that other methods can also be employed. An exemplary method that can be used is to employ antibodies that specifically bind to one or more of CD45, CXCR4, CD34, AC133, Sca-1, CD45R/B220, Gr-1, TCRaβ, TCRγδ, CD11b, and Ter-119, with the antibodies comprising a moiety (e.g., biotin) for which a high affinity binding reagent is available (e.g., avidin or streptavidin). For example, a biotin moiety could be attached to antibodies for each marker for which the presence on the cell surface is desirable (e.g., CD34, Sca-1, CXCR4), and the cell population with bound antibodies could be contacted with an affinity reagent comprising an avidin or streptavidin moiety (e.g., a column comprising avidin or streptavidin). Those cells that bound to the column would be recovered and further fractionated as desired. Alternatively, the antibodies that bind to markers present on those cells in the population that are to be removed (e.g., CD45R/B220, Gr-1, TCRaβ, TCRγδ, CD11b, and Ter-119) can be labeled with biotin, and the cells that do not bind to the affinity reagent can be recovered and purified further.

It is also understood that different separation techniques (e.g., affinity purification and FACS) can be employed together at one or more steps of the purification process.

In some embodiments, a VSEL stem cell or derivative thereof also expresses a marker selected from the group including but not limited to c-met, c-kit, LIF-R, and combinations thereof. In some embodiments, the disclosed isolation methods further comprise isolating those cells that are c-met⁺, c-kit⁺, and/or LIF-R⁺.

In some embodiments, the VSEL stem cell or derivative thereof also expresses SSEA-1, Oct-4, Rev-1, and Nanog, and in some embodiments, the disclosed isolation methods further comprise isolating those cells that express these genes.

In some embodiments, the population of CD133⁺/GlyA^{neg}/CD45^{neg} cells of the presently disclosed subject matter are further separated based on expression of aldehyde dehydrogenase (ALDH). For example, the ligand ALDEFLUOR® (STEMCELL Technologies, Vancouver, British Columbia, Canada) can be used to separate CD133⁺/GlyA^{neg}/CD45^{neg} cells based on ALDH staining. As such, the presently disclosed methods can in some embodiments further comprise isolating ALDH^{high} cells from the CD133⁺/GlyA^{neg}/CD45^{neg} cells, ALDH^{low} cells from the CD133⁺/GlyA^{neg}/CD45^{neg} cells, or both ALDH^{high} cells and ALDH^{low} cells separately from the CD133⁺/GlyA^{neg}/CD45^{neg} cells.

The presently disclosed subject matter also provides isolated populations of stem cells, wherein the isolated populations of stem cells comprises substantially purified CD133⁺/GlyA^{neg}/CD45^{neg} cells isolated from cord blood (CB). The isolated populations of stem cells can comprise CD133⁺/GlyA^{neg}/CD45^{neg}/ALDH^{high} cells, CD133⁺/GlyA^{neg}/CD45^{neg}/ALDH^{low} cells, or a combination thereof.

A population of cells containing the CD133⁺/CD45^{neg}/GlyA^{neg} cells of the presently disclosed subject matter can be isolated from any subject or from any source within a subject that contains them. In some embodiments, the population of cells comprises a bone marrow sample, a cord blood sample, a peripheral blood sample, or a fetal liver sample. In some embodiments, the population of cells is isolated from bone marrow of a subject subsequent to treating the subject with an amount of a mobilizing agent sufficient to mobilize the CD45^{neg} stem cells from bone marrow into the peripheral blood of the subject. As used herein, the phrase “mobilizing agent” refers to a compound (e.g., a peptide, polypeptide, small molecule, or other agent) that when administered to a subject results in the mobilization of a VSEL stem cell or a derivative thereof from the bone marrow of the subject to the peripheral blood. Stated another way, administration of a mobilizing agent to a subject results in the presence in the subject's peripheral blood of an increased number of VSEL stem cells and/or VSEL stem cell derivatives than were present therein immediately prior to the administration of the mobilizing agent. It is understood, however, that the effect of the mobilizing agent need not be instantaneous, and typically involves a lag time during which the mobilizing agent acts on a tissue or cell type in the subject in order to produce its effect. In some embodiments, the mobilizing agent comprises at least one of granulocyte-colony stimulating factor (G-CSF) and a CXCR4 antagonist (e.g., a T140 peptide; Tamamura et al. (1998) 253 Biochem Biophys Res Comm 877-882).

The presently disclosed subject matter also provides a population of CD45^{neg} stem cells isolated by the presently disclosed methods.

### III. Methods and Compositions for Administration to Subjects

III.A. Methods

The presently disclosed subject matter also provides methods for repopulating a cell type in a subject. In some embodiments, the methods comprise administering to the subject a composition comprising a plurality of isolated CD133⁺/GlyA^{neg}/CD45^{neg} stem cells in a pharmaceutically acceptable carrier in an amount and via a route sufficient to allow at least a fraction of the CD133⁺/GlyA^{neg}/CD45^{neg} stem cells to engraft a target site and differentiate therein, whereby a cell type is repopulated in the subject. In some embodiments, the cell type is a hematopoietic cell. In some embodiments of the presently disclosed methods, the plurality of isolated CD133⁺/GlyA^{neg}/CD45^{neg} stem cells comprises CD133⁺/GlyA^{neg}/CD45^{neg} stem cells isolated from cord blood. In some embodiments, the target site comprises the bone marrow of the subject.

Hence, in some embodiments the presently disclosed subject matter provides methods for bone marrow transplantation. In some embodiments, the methods comprise administering to a subject with at least partially absent bone marrow a pharmaceutical preparation comprising an effective amount of CD133⁺/GlyA^{neg}/CD45^{neg} stem cells isolated from a source of said cells (e.g., cord blood, bone marrow, peripheral blood, and/or fetal liver), wherein the effective amount comprises an amount of isolated CD133⁺/GlyA^{neg}/CD45^{neg} stem cells sufficient to engraft in the bone marrow of the subject.

Bone marrow transplantation is a technique that generally would be well known to one of ordinary skill in the art after review of the instant disclosure. Several U.S. and other patents and patent applications have been published which describe variations on the standard technique. Briefly, a subject that will receive bone marrow transplantation (BMT) typically undergoes a series of pre-treatments that are designed to prepare the bone marrow space to receive administered cells. These pre-treatments can include, but are not limited to treatments designed to suppress the recipient's immune system so that the transplant will not be rejected if the donor and recipient are not histocompatible as well as to create space within the bone marrow to allow the administered cells to engraft. An exemplary space-creating pre-treatment comprises exposure to chemotherapeutics that destroy all or some of the bone marrow and total body irradiation (TBI).

As such, the presently disclosed subject matter provides in some embodiments a method wherein a subject with at least partially absent bone marrow has undergone a pre-treatment to at least partially reduce the bone marrow in the subject. As used herein, the phrase “a subject with at least partially absent bone marrow” refers to a subject that has received either a myeloablative treatment or a myeloreductive treatment, either of which eliminates at least a part of the bone marrow in the subject. Myeloablative and myeloreductive treatments would be know to one of ordinary skill in the art, and can include immunotherapy, chemotherapy, radiation therapy, or combinations thereof.

III.B. Compositions

Once a subject has undergone an appropriate pre-treatment, if necessary, a composition comprising an isolated population of CD133⁺/GlyA^{neg}/CD45^{neg} stem cell of the presently disclosed subject matter is administered. In some embodiments, the composition comprises CD133⁺/GlyA^{neg}/CD45^{neg} stem cell in a pharmaceutically acceptable carrier (optionally, a carrier that is pharmaceutically acceptable for use in a human).

In some embodiments, freshly isolated CD133⁺/GlyA^{neg}/CD45^{neg} stem cells of the presently disclosed subject matter are administered, although frozen cells can also be employed. Methods for cryopreserving stem cells for administration to subject are known to one of ordinary skill in the art.

In some embodiments, the CD133⁺/GlyA^{neg}/CD45^{neg} stem cells of the presently disclosed subject matter are co-cultured in the presence of a feeder cell layer to enhance the efficiency with which the cells engraft the subject and/or produce blood cells in the subject. In some embodiments, the feeder cell layer comprises OP9 cells.

III.B.1. Formulations

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic methods and compositions of the presently disclosed subject matter can be used with additional adjuvants or biological response modifiers including, but not limited to, cytokines and other immunomodulating compounds.

III.B.2. Administration

Suitable methods for administration the compositions of the presently disclosed subject matter include, but are not limited to intravenous administration and delivery directly to the target tissue or organ. In some embodiments, the method of administration encompasses features for regionalized delivery or accumulation of the cells at a target site (e.g., the bone marrow). In some embodiments, the cells are delivered directly into the target site. In some embodiments, selective delivery of the cells of the presently disclosed subject matter is accomplished by intravenous injection of cells, where they home to the target site and engraft therein.

III.B.3. Dose

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

### IV. Other Applications

The presently disclosed subject matter also provides methods for inducing hematopoietic competency in CD133⁺/GlyA^{neg}/CD45^{neg} stem cell. As used herein, the phrase “hematopoietic competency” refers to an ability of a CD133⁺/GlyA^{neg}/CD45^{neg} stem cell (or a progeny cell thereof) to differentiate into a hematopoietic cell (e.g., a terminally differentiated hematopoietic cell). The phrase thus encompasses the efficiency at which an individual cell can repopulate a subject (e.g., as measured by the minimum number of cells that need to be administered to a subject in order for the subject to receive a clinically relevant benefit) as well as the time necessary for the cell to generate the clinically relevant benefit in the subject. In some embodiments, the hematopoietic competency of the cells of the presently disclosed subject matter comprises an ability to provide long term engraftment of the bone marrow in the subject.

As disclosed herein, CD133⁺/GlyA^{neg}/CD45^{neg} stem cells can show differing hematopoietic competencies based, in some embodiments, on the source from which the CD133⁺/GlyA^{neg}/CD45^{neg} stem cells were isolated, and any pre-treatment that the cells might have received (e.g., co-culture with OP9 cells). Therefore, in some embodiments the methods of the presently disclosed subject matter comprise (a) providing a CD133⁺/GlyA^{neg}/CD45^{neg} stem cell; and (b) co-culturing the CD133⁺/GlyA^{neg}/CD45^{neg} stem cell in the presence of a feeder layer (e.g., an OP9 feeder layer) for a time sufficient to induce hematopoietic competency in the CD133⁺/GlyA^{neg}/CD45^{neg} stem cell.

Additionally, the presently disclosed methods can employ the CD133⁺/GlyA^{neg}/CD45^{neg} stem cells that are bone marrow-derived CD133⁺/GlyA^{neg}/CD45^{neg} stem cells, cord blood-derived CD133⁺/GlyA^{neg}/CD45^{neg} stem cells, or combinations thereof. Additionally, the CD133⁺/GlyA^{neg}/CD45^{neg} stem cells are CD133⁺/GlyA^{neg}/CD45^{neg}/ALDH^{low} stem cells, CD133⁺/GlyA^{neg}/CD45^{neg}/ALDH^{high} stem cells, or a combination thereof.

### V. Cell Culture Systems

In some embodiments, the presently disclosed subject matter provides cell culture systems comprising CD133⁺/GlyA^{neg}/CD45^{neg} stem cells. In some embodiments, the cell culture systems further comprise a feeder cell layer, optionally an OP9 cell feeder layer.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods Employed in Examples 1-4

Recently, a primitive population of Very Small Embryonic-Like stem cells (VSELs) was identified in umbilical cord blood (CB). These CB-VSEL stem cells (i) are very small in size (<6 μm, typically 2-4 μm); (ii) are SSEA-4⁺/Oct-4⁺/CD133⁺/CXCR4⁺/Lin^{neg}/CD45^{neg}; (iii) respond robustly to a stroma derived factor-1 (SDF-1) gradient; and (iv) possess relatively large nuclei containing primitive euchromatin (Kucia et al. (2007) Leukemia 21:297-303; PCT International Patent Application Publication Nos. WO 2007/067280 and 2009/059032; the entire disclosures of which are incorporated herein by reference). Prior to the instant disclosure, the potential hematopoietic capacity of CB-derived CD133⁺/Lin^{neg}/CD45^{neg} VSELs was unknown.

Umbilical cord blood (CB) samples were collected from healthy donors. Red blood cells (RBCs) were removed by lysis employing hypotonic solution of ammonium chloride that results in the optimal recovery of CB-VSELs.

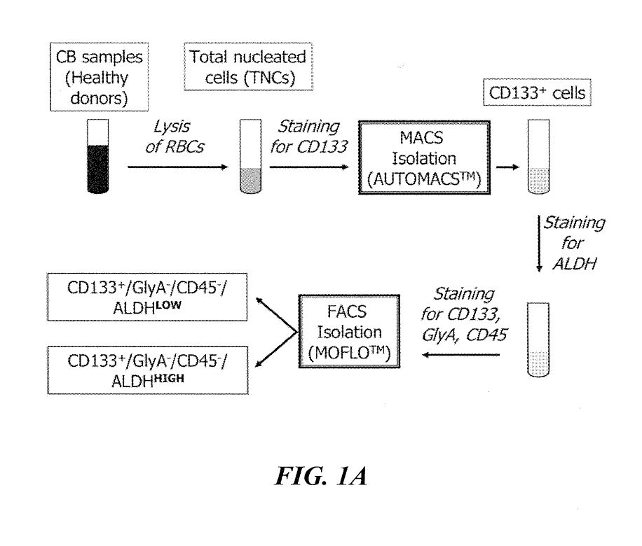

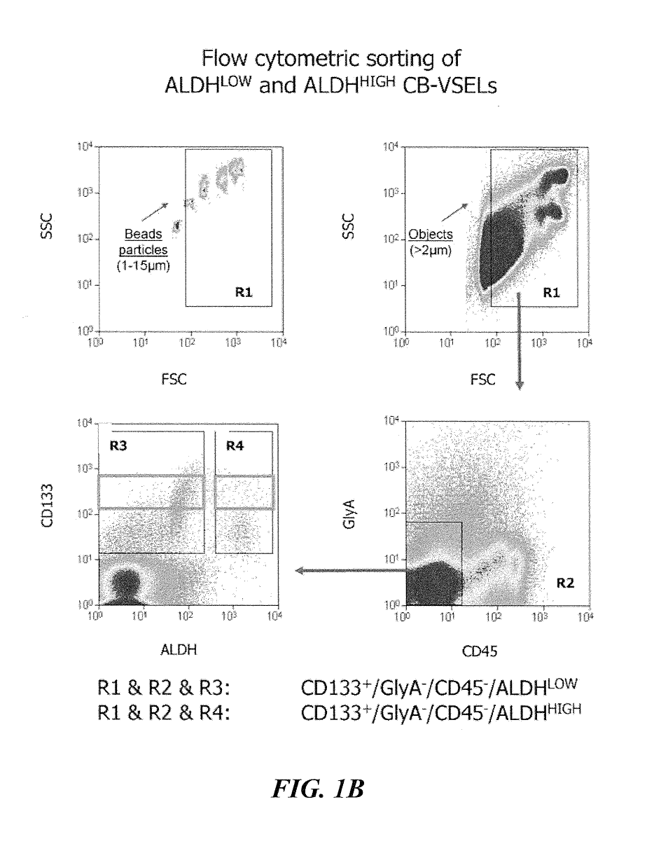

Total CB nucleated cells (TNCs) were stained for CD133 and then CD133⁺ cells were separated by magnetic cell sorting (MACS) performed with AUTOMACS™ system (Miltenyi Biotec Inc., Auburn, Calif., United States of America; see FIG. 1A).

CD133⁺ fraction was subsequently stained with ALDEFLUOR® reagent (STEMCELL Technologies, Vancouver, British Columbia, Canada) detecting ALDH followed by immunolabeling of CD45 and Glycophorin A (GlyA) as well as re-staining of CD133 for further separation. CD133⁺/GlyA^{neg}/CD45^{neg}/ALDH^{low} and CD133−/GlyA^{neg}/CD45^{neg}/ALDH^{high} CB-VSEL subpopulations were separated by fluorescence activated cell sorting (FACS) by employing a MOFLO™ sorter (Beckman Coulter, Inc. Miami, Fla., United States of America; see FIG. 1B).

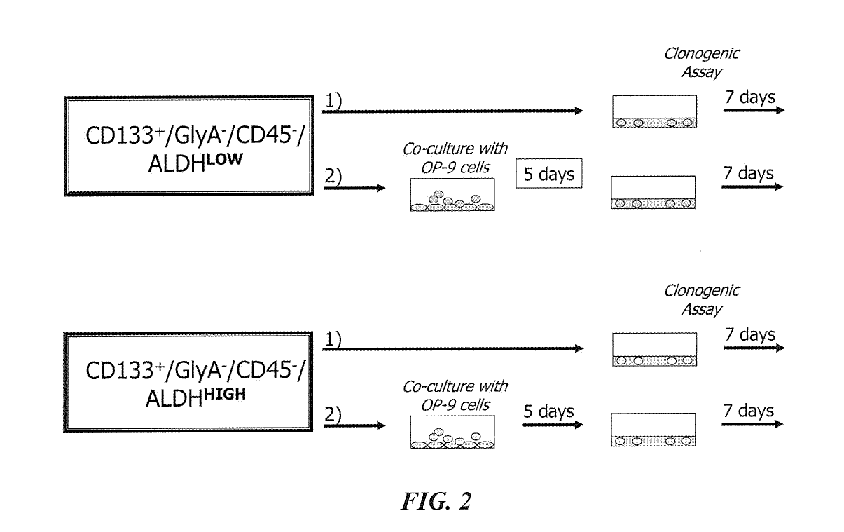

In the first step, both freshly isolated fractions of CB-VSELs were tested by clonogenic assay in methylcellulose supplemented with hematopoietic growth factors (IL-3, GM-CSF, SCF, EPO, Flt-3 and TPO) to identify hematopoietic capacity. Next, both subpopulations of CB-VSELs were cultured over OP9 stroma cells for 5 days and subsequently transferred to methylcellulose supplemented with growth factors. The number of colonies was calculated after 7 days of culture (see FIG. 2).

The expression levels of genes related to pluripotency or hematopoietic commitment (Oct-4, C-myb, HoxB-4, and LMO-2) were determined in both freshly isolated CB-VSELs and CB-VSEL-derived cells expanded over OP9 cells by real time RT-PCR.

### Example 1

### Freshly Isolated CB-VSELs do not Exhibit Hematopoietic Potential but can Become Hematopoietic after Co-Culture Over OP9 Cells

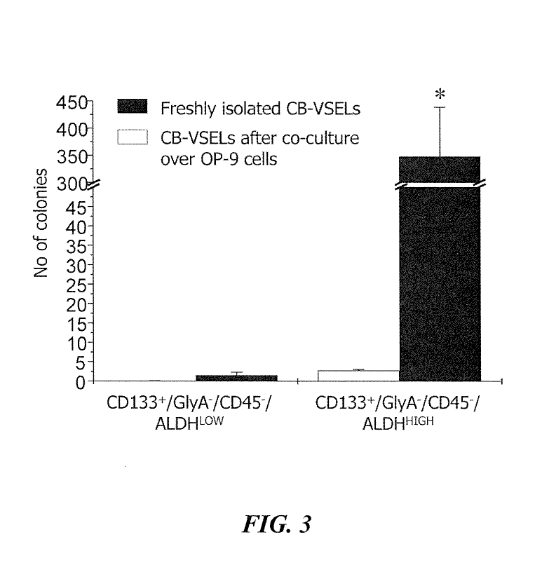

Clonogenic assays were employed to test the hematopoietic potential of freshly isolated CB-VSELs in vitro. Neither freshly isolated CD133⁺/GlyA^{neg}/CD45^{neg}/ALDH^{low} nor CD133⁺/GlyA^{neg}/CD45^{neg}/ALDH^{high} CB-VSELs were able to grow hematopoietic colonies in vitro (see FIG. 3).

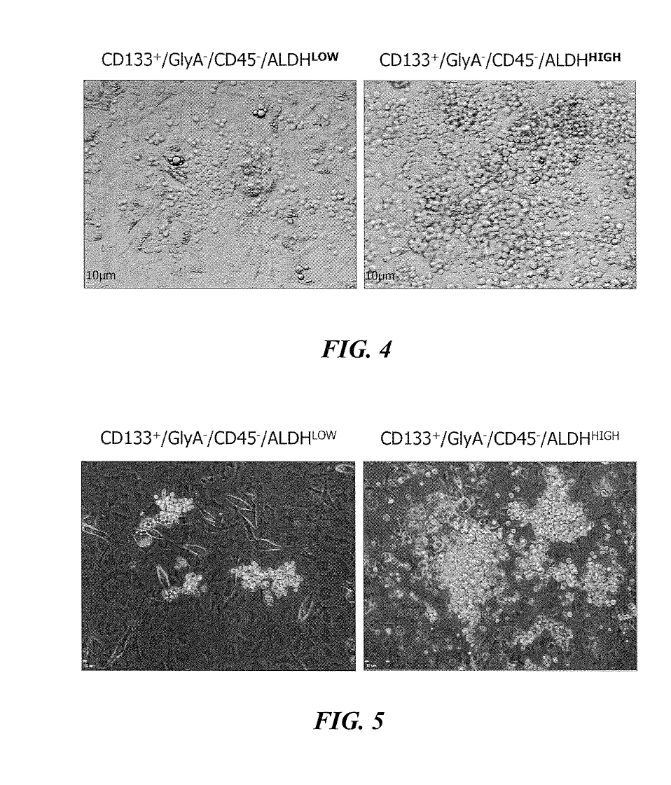

However, when either fraction (i.e., ALDH^{low} or ALDH^{high}) of freshly isolated CD133⁺/GlyA^{neg}/CD45^{neg} CB-VSELs were co-cultured over OP9 stromal cells, they acquired in vitro hematopoietic potential (see FIGS. 3 and 4). Both ALDH^{low} and ALHD^{high} CD133⁺/GlyA^{neg}/CD45^{neg} CB-VSELs formed primitive colonies resembling “cobble-stone” areas, which is typical for long term hematopoietic stem cells (LT-HSCs; see FIG. 4). Interestingly, ALDH^{high} CB-VSELs formed such colonies more quickly that ALDH^{kow} CB-VSELs did.

Cells expanded over OP9 feeder layer were subsequently transferred into methylcellulose supplemented with hematopoietic growth factors. A significant increase in colony formation by CD133⁺/GlyA^{neg}/CD45^{neg}/ALDH^{high}-derived cells was observed as compared to the CD133⁺/GlyA^{neg}/CD45^{neg}/ALDH^{low}-derived population. Here as well, the clonogenic activity of the latter cells was delayed in time (see FIG. 3). Representative brightfield images of such colonies obtained from both fractions are shown on FIG. 5.

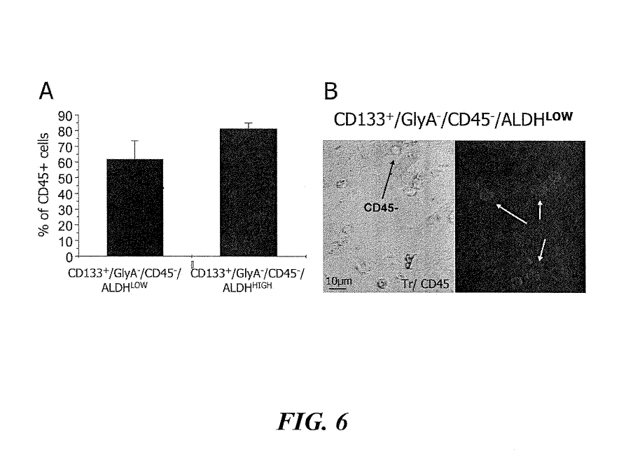

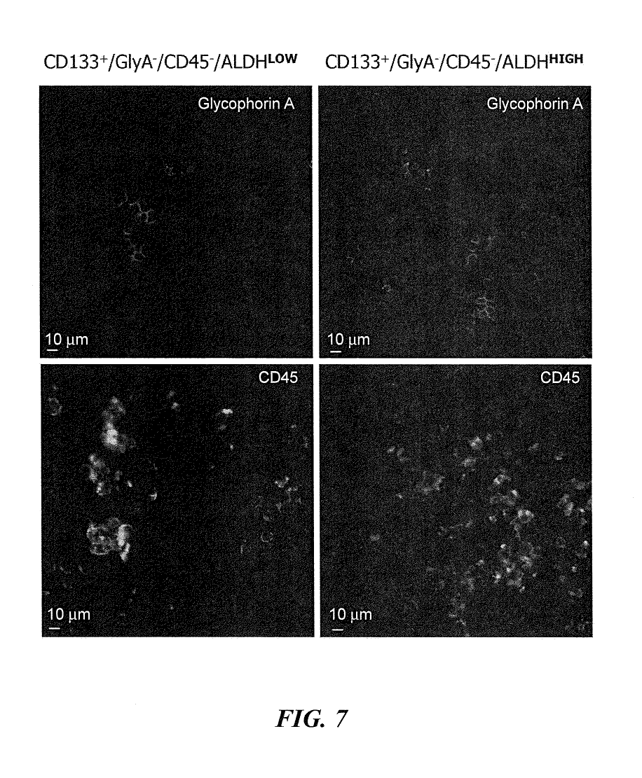

Flow cytometric and epifluorescence microscopic analyses revealed that cells harvested from colonies initiated by CD133⁺/GlyA^{neg}/CD45^{neg}/ALDH^{low} and CD133⁺/GlyA^{neg}/CD45^{neg}/ALDH^{high} CB-VSELs acquired expression of CD45 (see FIG. 6). Similarly, hematopoietic colonies initiated from both subpopulations of CD133⁺/GlyA^{neg}/CD45^{neg} CB-VSELs stained positively for several hematopoietic markers, including GlyA and CD45 (see FIG. 7).

### Example 2

### CD133⁺/GlyA^{neg}/CD45^{neg}/ALDH^{low} CB-VSELs are Enriched in Primitive Subpopulations of Cells Expressing Markers of Pluripotent Stem Cells

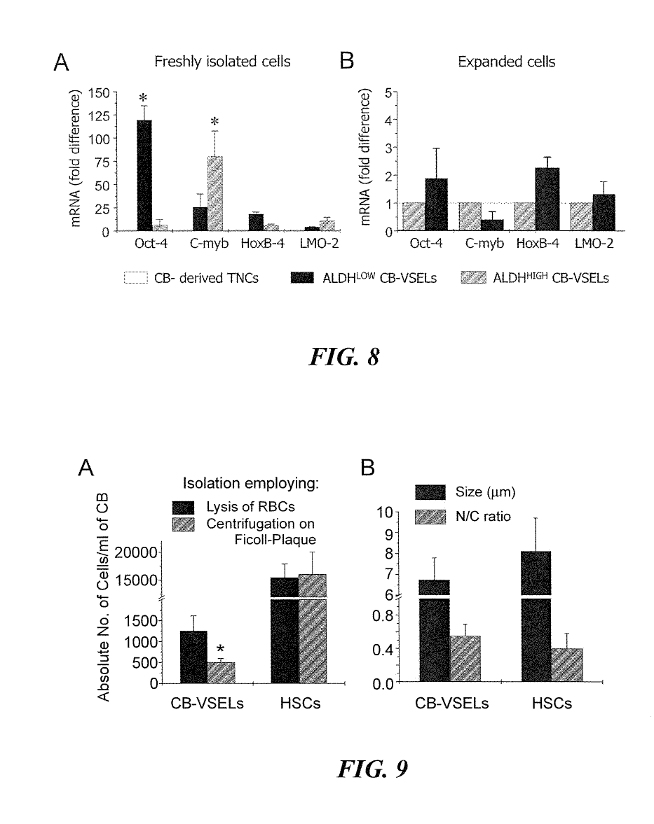

By employing real time RT-PCR analysis, it was determined that freshly isolated CD133⁺/GlyA^{neg}/CD45^{neg}/ALDH^{low} CB-VSELs exhibited a 119.5±15.5 fold difference higher level of mRNA for the exemplary pluripotent stem cells marker Oct-4 as compared to CB-derived TNCs (see FIG. 8A). The CD133⁺/GlyA^{neg}/CD45^{neg}/ALDH^{high} subpopulation of CB-VSELS expressed higher levels of genes related to hematopoiesis such as C-myb (80.2±27.4 fold difference when compared to CB-derived TNCs; see FIG. 8A).

After co-culture over OP9 cells, the expression of Oct-4 declined in ALDH^{low} CB-VSELs (only 1.9±1.1 fold difference as compared to ALDH^{high} CB-VSELs), while the expression of several hematopoietic genes increased (see FIG. 8B).

### Example 3

### Loss of CB-VSELs Occurs During Routine Processing of CB Units

By employing flow cytometric analyses, it was determined that a significant portion (42.5±12.6%) of CD133⁺/Lin^{neg}/CD45^{neg} CB-VSELs can be lost during routine preparation of CB units for storage and/or freezing. A similar effect was also observed after centrifugation over a Ficoll-Paque gradient (see FIG. 9A), perhaps due to the unusually small size and high density of CB-VSELs. FIG. 9B shows that CB-VSELs were characterized by smaller size and higher N/C ratio than HSCs by IMAGESTREAM™ system analysis.

### Example 4

### Contribution of CB-Derived VSELs to Hematopoietic Lineages in Engrafted NOD/SCID Mice

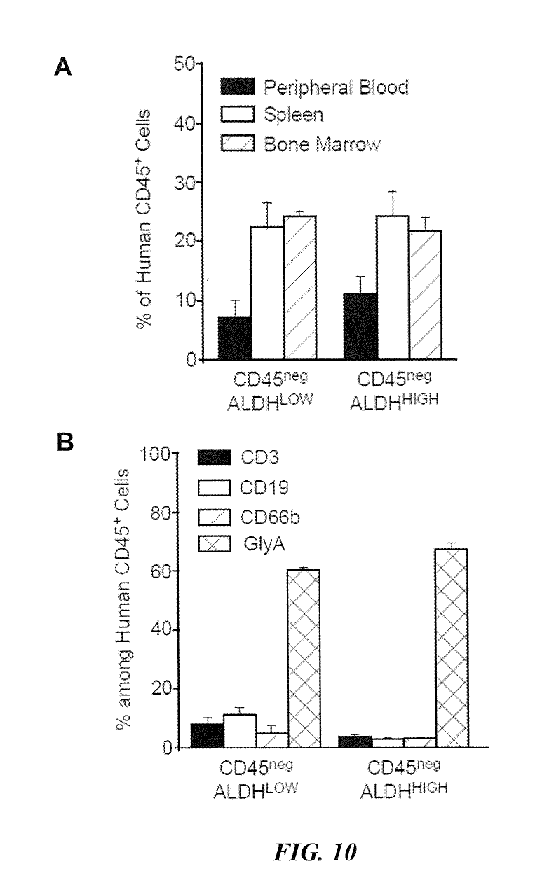

The hematopoietic potential of CB-derived VSELs was tested in vivo after transplantation into lethally irradiated NOD/SCID mice (see FIGS. 10A and 10B).

Both CD45^{neg}/CD133⁺/ALDH^{high} and CD45^{neg}/CD133⁺/ALDH^{low} VSELs gave rise to human lympho-hematopoietic chimerism in lethally irradiated NOD/SCID mice assayed 4-6 weeks after transplantation. The level of human hematopoietic CD45⁺ cells in murine peripheral blood (PB), bone marrow (BM), and spleen (SP) were comparable in both transplanted CB-VSELs fractions: 7.1±2.9% in PB, 23.2±0.2% in SP, and 25.2±1.0% in BM. This data suggested that freshly isolated CD45^{neg} CB-VSELs were depleted from clonogeneic progenitors, but were highly enriched for primitive HSCs.

Based on the in vitro and in vivo data disclosed herein, the following hierarchy of hematopoietic stem cells in CB from more primitive to more differentiated was apparent: CD45^{neg}/CD133⁺/ALDH^{low}; CD45^{neg}/CD133⁺/ALDH^{high}; CD45⁺/CD133⁺/ALDH^{low}; and hen CD45^{neg}/CD133⁺/ALDH^{high}. The data presented herein also suggested that human CB-derived CD45^{neg} VSELs represented a population of very primitive long term repopulating HSCs (LT-HSCs).

And finally, it was determined that currently employed routine CB processing strategies can result in the undesirable loss of up to about 50% of CB-VSELs, suggesting that such strategies negatively impact the overall efficiency of CB isolates as sources for LT-HSCs.

### Discussion of Examples 1-4

ALDH^{low} and ALDH^{high} CD133⁺/GlyA^{neg}/CD45^{neg} CB-VSELs became hematopoietic when expanded/co-cultured over OP9 stroma cells. Both fractions formed “cobble-stone” areas that contain cells capable to grow hematopoietic colonies.

The CD133⁺/GlyA^{neg}/CD45^{neg}/ALDH^{low} fraction of CB-VSELs was enriched in markers of pluripotent stem cells and exhibited delayed clonogenic capacity that was prolonged and sustained during in vitro cultures.

The CB processing procedures based on depletion of red blood cells (RBCs) by centrifugation on Ficoll-Paque gradient or volume reduction prior to storage/freezing can lead to significant loss of CB-VSELs.

CD133⁺/GlyA^{neg}/CD45^{neg}/ALDH^{low} very small CB-derived MNCs, expressing VSELs markers and exhibiting low activity of ALDH, were enriched for the most primitive population of LT-HSCs.

This population can play a role in long term engraftment of CB-derived cells and can provide a source of cells that can be employed for HSCs expansion.

### Materials and Methods for Examples 5-8

Animals. These disclosed experiments have been performed in accordance with the guidelines of the Laboratory Institutional Animal Care and Use Committee (IACUC) of the University of Louisville, Louisville, Ky., United States of America, and conforms to the Guide for the Care and Use of Laboratory Animals published by the U.S. National Institutes of Health (NIH Publication No. 85-23, revised 1996).

Isolation of FL cells for FACS sorting and analysis. Fetal liver cells were isolated from embryos of C57BL/6 mice (Jackson Laboratory, Bar Harbor, Me., United States of America) at 12.5 days post coitus (dpc), 15.5 dpc, and 17.5 dpc. Fetal livers from 15-20 fetuses were combined in each experiment. Tissue was mechanically fragmented and released cells were washed and filtered through 40 μm strainer. Red blood cells were subsequently lysed using 1×BD PHARMLYSE™ (BD PHARMINGEN™, San Jose, Calif., United States of America). The total number of nucleated cells obtained from one liver was calculated using a hemocytometer and was applied for computing absolute numbers of populations detected in liver. Freshly isolated cells were further assayed for the expression of CD45, hematopoietic lineages markers (Lin), and Sca-1 for 30 minutes in medium containing 2% of betal bovine serum (FBS). The following rat anti-mouse antibodies (obtained from BD PHARMINGEN™, San Jose, Calif., United States of America) were used to stain isolated cells: anti-CD45 (clone 30-F11; conjugated to APC-Cy7™, a dual fluorochrome composed of allophycocyanin (APC) coupled to the cyanine dye Cy7™), anti-CD45R/B220 (clone RA3-6B2, conjugated to phycoerythrin (PE)), anti-Gr-1 (clone RB6-8C5, conjugated to PE), anti-TCRαβ (clone H57-597, conjugated to PE), anti-TCRγδ (clone GL3, conjugated to PE), anti-CD11b (clone M1/70, conjugated to PE), anti-Ter119 (clone TER-119, conjugated to PE) and anti-Ly-6A/E (Sca-1; clone E13-161.7, conjugated to biotin and detected with streptavidin conjugated with PE-Cy5™). Isotype controls were used to estimate the positive populations. After staining, the cells were washed, re-suspended in RPMI medium with 10% FBS, and sorted using a MOFLO™ cell sorter (Beckman Coulter, Inc., Miami, Fla., United States of America).

Sorting was performed with a rate of sorted events between 5000 and 10,000 cells/sec according to the previously described strategy for isolation of VSELs from murine bone marrow (Zuba-Surma et al. (2008) J Cell Mol Med 12:292-303). Briefly, cells were visualized in a first step by dot plot showing forward scatter (FSC) vs. side scatter (SSC) signals, which were related to the size and granularity/complexity of the cell, respectively. Agranular, small events ranging from 2-10 μm were selected for sorting after comparison with six differently sized beads particles with standard diameters of 1, 2, 4, 6, 10 and 15 μm (Flow Cytometry Size beads available from INVITROGEN™, a division of Life Technologies Corp., Carlsbad, Calif., United States of America). These small cells were analyzed for expression of Sca-1 and Lineage markers, and Sca-1⁺/Lin^{neg} events were included for sorting and further separation according to CD45 expression into two populations: Sca-1⁺/Lin^{neg}/CD45^{neg} cells (VSELs) and Sca-1⁺/Lin^{neg}/CD45⁺ cells (HSCs). See Zuba-Surma et al. (2008) J Cell Mol Med 12:292-303.

IMAGESTREAM® System (ISS) analysis. Fetal liver tissue was isolated and processed as described hereinabove for FACS sorting and analysis. Briefly, the full population of nucleated FL-derived cells was obtained after mechanical digestion of tissue and further lysis of red blood cells (RBCs) using 1×BD PHARMLYSE™ Buffer (BD PHARMINGEN™). Cells were subsequently stained for CD45 expression, expression of Lin markers, and expression of the Sca-1 antigen. Based on the detection channels available for the ISS, the following anti-mouse antibodies were employed for staining: rat anti-CD45 (PE-Cy5™-conjugated clone 30-F11; eBioscience, San Diego, Calif., United States of America), “lineage cocktail” (BD PHARMINGEN™, San Jose, Calif., United States of America, which includes anti-CD45R/B220 (PE-conjugated clone RA3-6B2); anti-Gr-1 (PE-conjugated clone RB6-8C5), anti-TCRαβ (PE-conjugated clone H57-597), anti-TCRγδ (PE-conjugated clone GL3), anti-CD11b (PE-conjugated clone M1/70), anti-Ter119 (PE-conjugated clone TER-119)); and anti-Ly-6A/E (Sca-1; fluorescein isothiocyanate (FITC)-conjugated clone E13-161.7; BD PHARMINGEN™). Cells were washed after staining, fixed with 4% paraformaldehyde for 20 minutes, and permeabilized with 0.1% TRITON® X-100 solution for 10 minutes. 7-aminoactinomycin D (7-AAD; INVITROGEN™; 40 μM) was added 5 minutes before analysis to visualize nuclei, and samples were further acquired and analyzed using an IMAGESTREAM® System 100 (Amnis Corporation, Seattle, Wash., United States of America). See Basiji et al. (2007) Clin Lab Med 27:653-670; Zuba-Surma et al. (2007a) Folia Histochem Cytobiol 45:279-290; Zuba-Surma et al. (2007b) Adv Cell Biol 34:361-375.

For identification of an SSEA-1⁺/Sca-1⁺/Lin^{neg}/CD45^{neg} subpopulation that positively stained for the embryonic surface marker SSEA-1, cells were initially incubated in the presence of 10% donkey serum (Jackson Immunoresearch, West Grove, Pa., United States of America) to block sites of non-specific binding of secondary antibodies followed by staining with primary anti-murine SSEA-1 antibody (murine IgM; Chemicon Int., Temecula, Calif., United States of America; 1:200) for 2 hours at 37° C. A secondary antibody conjugated to FITC (polyclonal donkey anti-mouse IgM; Jackson Immunoresearch) was added after washing. Cells were incubated for 2 hours at 37° C. and subsequently washed and stained with directly conjugated antibodies against Sca-1 (PE-Cy5™), CD45 (PE), and Lin (PE). Stained cells were resuspended in PBS for further analysis. 7-AAD was added for 5 minutes before analysis and samples were run directly on the ISS 100.

For intranuclear Oct-4 detection and identification of the Oct-4⁺/Sca-1⁺/Lin^{neg}/CD45^{neg} population, freshly isolated cells were initially fixed with 4% paraformaldehyde for 20 minutes and then permeabilized with a 0.1% TRITON® X-100 solution for 10 minutes. Cells were washed and incubated in the presence of 10% donkey serum (Jackson Immunoresearch) and stained with primary anti-murine Oct-4 antibody (mouse monoclonal IgG; Chemicon Int.; 1:200) for 2 hours at 37° C. A secondary antibody conjugated to FITC (polyclonal donkey anti-mouse IgG; Jackson Immunoresearch) was added following washing. Cells were incubated for 2 hours at 37° C. Following the staining for Oct-4, cells were incubated with directly conjugated antibodies against Sca-1 (PE-Cy5), CD45 (PE), and Lin (PE). Stained cells were resuspended in PBS for further analysis. 7-AAD was added for 5 minutes before analysis and samples were run directly on the ISS 100.

Signals from FITC, PE, 7-AAD, and PE-Cy5 were detected by channels 3, 4, 5 and 6, respectively, while side scatter and brightfield images were collected in channels 1 and 2, respectively.

Expansion and VSELs-DS formation culture. Freshly sorted Sca-1⁺/Lin^{neg}/CD45^{neg} (VSEL) and Sca-1⁺/Lin^{neg}/CD45⁺ (HSC) cells were cultured over C2C12 murine myoblast feeder layers seeded on 22 mm glass-bottom plates (Willco Wells B.V., Amsterdam, Netherlands). Cells were cultured in medium containing a low percentage of serum (DMEM with 2% FBS, INVITROGEN™) without any supplementing growth factors. VSEL-derived sphere (VSELs-DS) formation was estimated after 9 days of culture by counting.

Real time PCR. The expression at the level of mRNA of markers of liver lineage commitment (α-fetoprotein and cytokeratin 19; CK19) and markers associated with cellular pluripotency (Oct-4, Nanog, Rex-1, Dppa1, and Rif1) was investigated in freshly isolated Sca-1⁺/Lin^{neg}/CD45^{neg} (VSEL) and Sca-1⁺/Lin^{neg}/CD45⁺ (HSC) as compared to unfractionated FL-derived cells. Total mRNA was isolated with the RNeasy Mini Kit (Qiagen Inc., Valencia, Calif., United States of America) and reverse-transcribed with TAQMAN® Reverse Transcription Reagents (Applied Biosystems, Inc., Foster City, Calif., United States of America). Quantitative assessments of mRNA expression of the genes of interest and of β2-microglobulin were performed by real-time RT-PCR using an ABI PRISM® 7000 Sequence Detection System (Applied Biosystems, Inc.). The primers were designed with PRIMER EXPRESS® software and previously published. See Kucia et al. (2006) Leukemia 20:857-869. A 25 μl reaction mixture containing 12.5 μl of SYBR® Green PCR Master Mix (Applied Biosystems, Inc.) and 10 ng of forward and reverse primers was used. The threshold cycle (Ct), defined as the cycle number at which the amount of amplified gene of interest reached a fixed threshold, was subsequently determined. Relative quantization of mRNA expression was calculated with the comparative Ct method. The relative quantitative value of target, normalized to an endogenous control β2-microglobulin gene and relative to a calibrator, was expressed as 2^{-ΔΔCt} (fold difference), where ΔCt=Ct of target genes (α-fetoprotein, CK19, Oct-4, Nanog, Rex-1, Dppa3, and Rif-1)−Ct of endogenous control gene (β2-microglobulin), and ΔΔCt=ΔCt of samples for target gene−ΔCt of calibrator for the target gene. To avoid the possibility of amplifying contaminating DNA, (i) all of the primers for real-time RT-PCR were designed containing an intron sequence for specific cDNA amplification; (ii) reactions were performed with appropriate negative controls (template-free controls); (iii) a uniform amplification of the products was rechecked by analyzing the melting curves of the amplified products (dissociation graphs); and (iv) the melting temperature (Tm) was 57-60° C. and the probe Tm was at least 10° C. higher than primer Tm.

Statistical methods. All values are presented as Mean±standard error of the mean (SEM). The percentage of different cellular populations in the fetal liver, the number of embryonic bodies formed, and the quantitative mRNA data (fold change in mRNA levels) were analyzed with one-way ANOVA. If the ANOVA showed an overall difference, post hoc contrasts were performed using the student t test for unpaired data. Probability (p) values of less than 0.05 were considered statistically significant. All statistical analyses were performed using Origin software (version 5.0, Microcal Software, Inc. Northampton, Mass., United States of America).

### Introduction to Examples 5-8

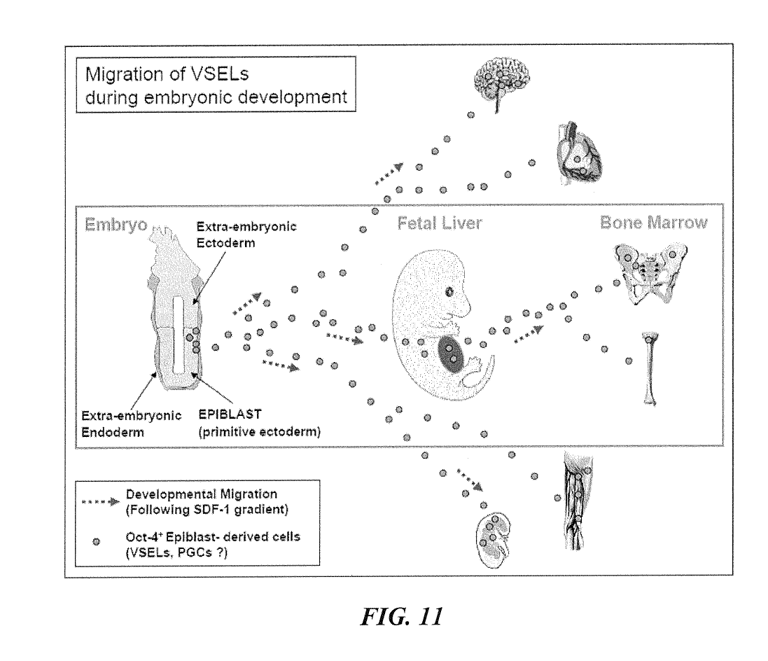

A population of very small Sca-1⁺/Lin^{neg}/CD45^{neg} cells has been identified in murine adult tissues including BM that express CXCR4 receptor and SSEA-1 antigen on their surface and early transcriptional factor Oct-4 in nuclei. The instant co-inventors have postulated that these cells are epiblast-derived pluripotent stem cells (PSCs) that are deposited in developing organs and survive into adulthood as a backup source of tissue committed stem cells (TCSCs) for various organs and tissues. They have also hypothesized that a significant fraction of these cells migrates along with HSCs to the FL, where by the end of second trimester of gestation in SDF-1-dependent manner they relocate from the FL to the developing BM microenvironment (see FIG. 11). Thus an aspect of EXAMPLES 5-8 was to investigate if the cells with VSELs characteristics are detectable in murine FLs isolated at different time of gestation (12.5, 15.5 and 17.5 dpc).

### Example 5

### Sca-1⁺/lin^{neg}/CD45^{neg} Cells in the Fetal Liver

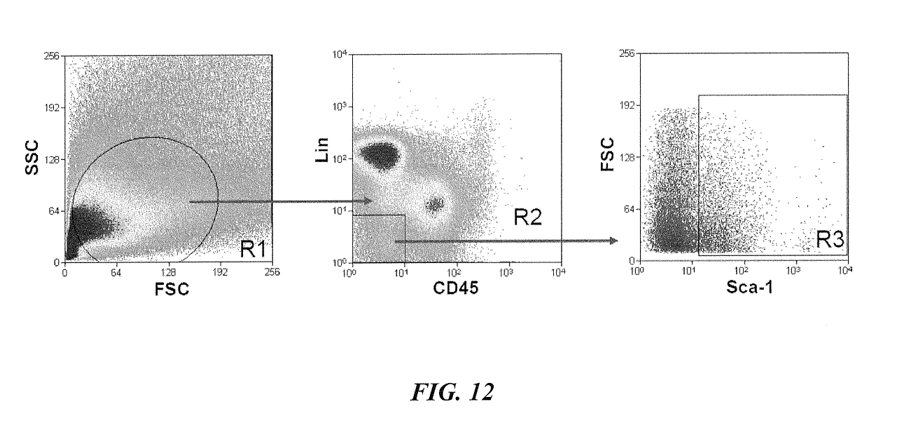

Flow cytometric analyses were employed to determine whether FL includes VSELs, and if so, to estimate the number of these cells in FL using the gating strategy depicted in FIG. 12. Briefly, murine FL-derived cells were isolated by enzymatic digestion, stained using antibodies for CD45 (APC-Cy7™) lineage markers (PE) and Sca-1 (PE-Cy5™), and analyzed with MOFLO™ as described hereinabove. The region that contained events between 2-10 μm (region R1 in FIG. 12) in size was designed by employing sized beads particles as described in Zuba-Surma et al. (2008) J Cell Mol Med 12:292-303. The cells from R1 were subsequently evaluated for expression of CD45 and also expression of lineage (Lin) markers, and Lin^{neg}/CD45^{neg} small events (region R2 in FIG. 12) were further analyzed for a presence of Sca-1 antigen. Region 3 (R3 in FIG. 12) enclosed Sca-1⁺ cells exhibiting the VSELs surface phenotype (Sca-1⁺/Lin^{neg}/CD45^{neg}).

Table 1 summarizes the percentages of various subpopulations at 12.5, 15.5 and 17.5 dpc. The values presented represent average numbers obtained from three independent experiments (Mean±SEM). Fetal livers from 15-20 fetuses were combined in each experiment.

As shown therein, the percentages of small Sca-1⁺/Lin^{neg}/CD45^{neg} cells decreased from 1.33±0.02% to 0.63±0.27% to 0.09±0.03% of total FL mononuclear cells at these time points (p<0.05 between day 12.5 and 17.5). At 17.5 dpc, the concentration of these cells reached the level observed in adult liver (see Zuba-Surma et al. (2008) Cytometry A 73A:1116-1127). In parallel, the percentages of cells present in FL with hematopoietic potential (i.e., CD45⁺ and Sca-1⁺) as well as cells that were Sca-1⁺/Lin^{neg}/CD45⁺ (i.e., cells that were enriched in HSCs) were also determined. The percentages of these cells also decreased, particularly between 15.5 and 17.5 dpc.

TABLE 1
Percentages of Various FL Cell Subpopulations Identified by FACS

| | Percent of Total FL cells (Mean ± SEM | | |
|---|---|---|---|
| Population | 12.5 dpc | 15.5 dpc | 17.5 dpc |
| CD45⁺ | 18.55 ± 2.55 | 19.10 ± 7.90 | 9.80 ± 4.20 |
| Sca-1⁺ | 19.95 ± 1.25 | 16.15 ± 7.65 | 4.20 ± 1.30 |
| Sca-1⁺/Lin^{neg}/CD45^{neg} | 1.33 ± 0.02 | 0.63 ± 0.27 | 0.09 ± 0.03 |
| (VSELs) | | | |
| Sca-1⁺/Lin^{neg}/CD45⁺ | 14.20 ± 1.50 | 10.05 ± 2.85 | 2.81 ± 1.11 |
| (HSCs) | | | |

(*) p < 0.05

### Example 6

### FL-Derived Sca-1⁺/Lin^{Neg}/CD45^{Neg} Cells Express Several PSCs Markers and Grow Spheres in Co-Cultures with C2C12 Myoblasts

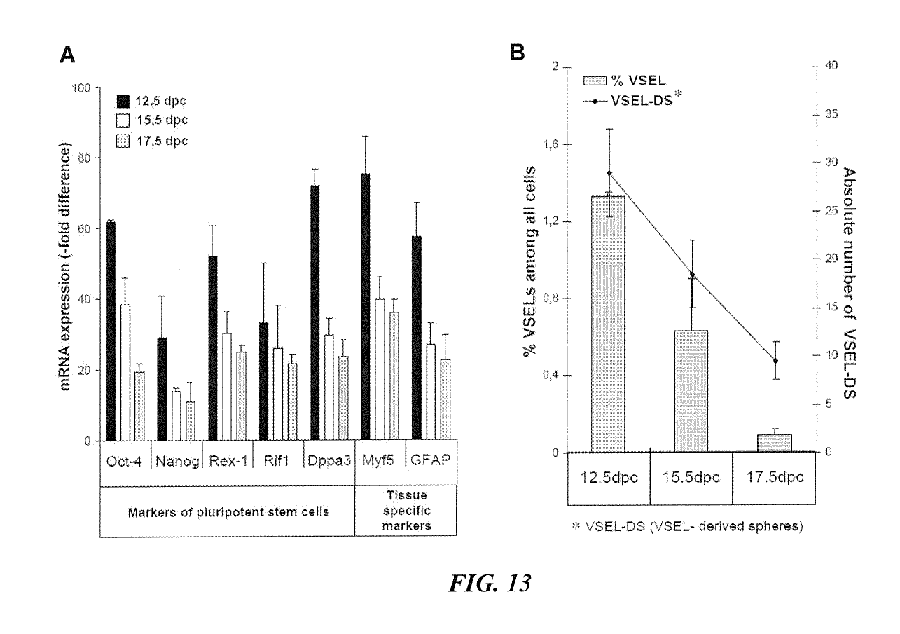

BM-derived VSELs express a multitude of PSCs markers, including Oct-4, Nanog, and Rex-1, and when cultured in the presence of a feeder layer composed of cells of the myoblastic cell line (C2C12) form characteristic fetal alkaline phosphatase-positive spheres resembling embryonic bodies. Thus, whether FL-derived Sca-1⁺/Lin^{neg}/CD45^{neg} cells expressed markers of PSCs and grow characteristic spheres in vitro was tested. The results are presented in FIG. 13.

In order to confirm the presence of pluripotent VSELs in FL, Sca-1⁺/Lin^{neg}/CD45^{neg} cells were sorted as described hereinabove, and the expression of genes of pluripotency at mRNA level was determined by real time RT-PCR. FIG. 13A shows that FL-derived Sca-1⁺/Lin^{neg}/CD45^{neg} VSELs expressed all of these pluripotency genes as compared to FL-derived mononuclear cells. The level of mRNA for Oct-4, Nanog, Rex-1, Dppa-1, and Rif1 was 61.64±9.67, 28.88±11.80, 51.86±8.65, 71.82±10.67, and 33.17±4.68 fold higher, respectively, in Sca-1⁺/Lin^{neg}/CD45^{neg} cells than in unfractionated FL mononuclear cells. These cells also highly expressed Myf5 and GFAP, which are early mesodermal and ectodermal transcription factors. A decrease in expression of all of these genes was also observed with the age of embryo, showing the highest level of expression at 12.5 dpc.

Next, whether FL-derived Sca-1⁺/Lin^{neg}/CD45^{neg} cells generated spheres and if their number depended on the age of murine embryo was investigated. It was determined that cells sorted by FACS from FL Sca-1⁺/Lin^{neg}/CD45^{neg} cells cultured over C2C12 supportive cell line grew spheres, while Sca-1⁺/Lin^{neg}/CD45⁺ HSCs did not (see FIG. 13B). Moreover, the number of spheres decreased with increasing embryonic age, showing the highest number at 12.5 dpc and decreasing at 15.5 and 17.5 dpc (see FIG. 13B).

### Example 7

### IMAGESTREAM™ Analyses of FL-Derived Sca-1⁺/Lin^{Neg}/CD45^{Neg} Cells

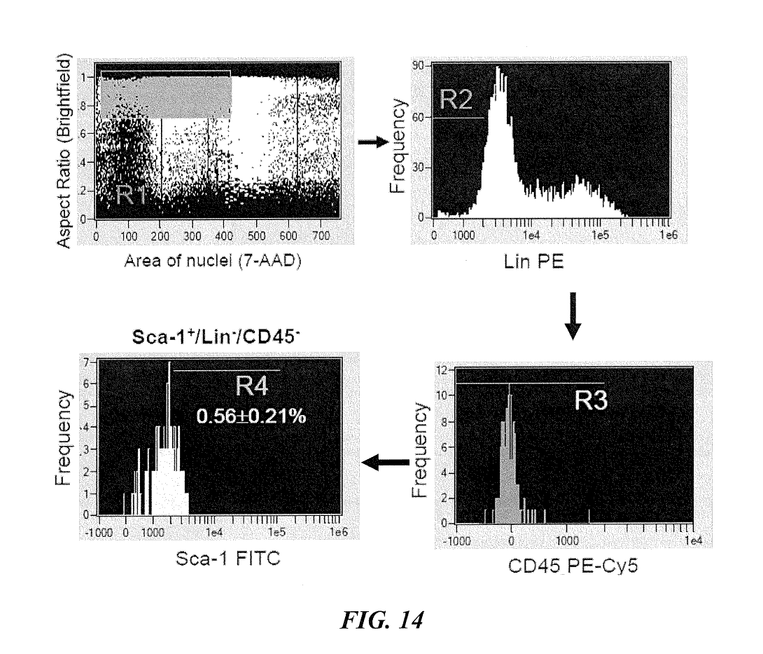

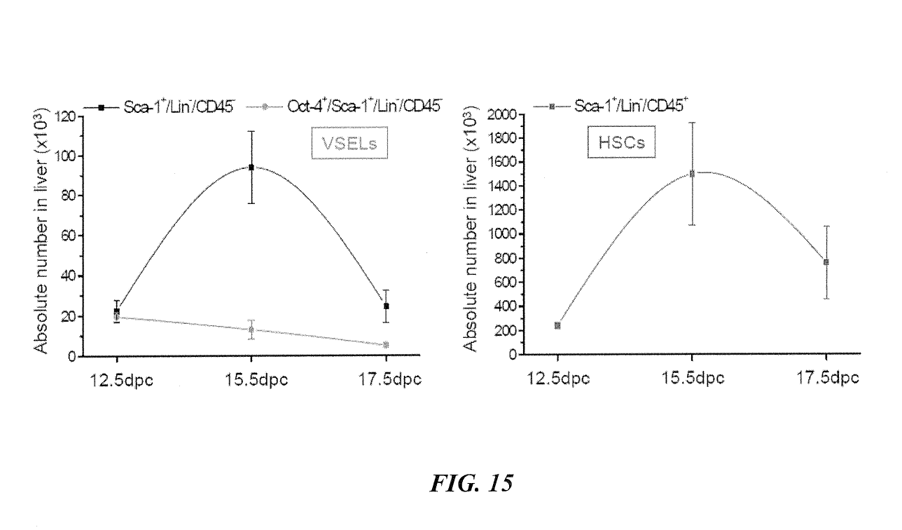

IMAGESTREAM™ analyses were employed to assess the average size and nuclear cytoplasmic (N/C) ratio of FL-derived Sca-1⁺/Lin^{neg}/CD45^{neg} VSELs compared to FL-derived Sca-1⁺/Lin^{neg}/CD45⁺ HSCs. The results are presented in FIG. 14. As shown therein, it was determined that FL-derived VSELs and HSCs were 7.19±0.10 μm and 9.44±0.07 μm in diameter, respectively. Thus, the average diameter of Sca-1⁺/Lin^{neg}/CD45^{neg} cells isolated from FL was about 50% higher than that of Sca-1⁺/Lin^{neg}/CD45^{neg} VSELs isolated from the adult BM (Zuba-Surma et al. (2008) J Cell Mol Med 12:292-303).

N/C ratio was calculated as nuclear area divided by cytoplasmic area computed from nuclear (identified by 7-AAD staining) and brightfield images. The values represent average numbers obtained from three independent experiments (Mean±SEM). Fetal livers from 15-20 fetuses were combined in each experiment. The N/C ratio for FL-derived VSELs and HSCs was calculated as 2.63±0.48 and 1.77±0.13, respectively (see Table 2), which is similar to that found in BM.

TABLE 2
Sizes and N/C Ratios of FL-derived VSELs and HSCs

| Population | Content (%) | Size (μm) | N/C Ratio |
|---|---|---|---|
| Sca-1⁺/Lin^{neg}/CD45^{neg} | 0.56 ± 0.21 | 7.19 ± 0.10 | 2.63 ± 0.48 |
| Sca-1⁺/Lin^{neg}/CD45⁺ | 6.47 ± 0.72 | 9.44 ± 0.07 | 1.77 ± 0.13 |

Two different populations of Sca-1⁺/Lin^{neg}/CD45^{neg} cells were distinguished according to their size: smaller or larger than 6 μm. ISS analyses of cells from both subfractions were performed with respect to expression of Sca-1, hematopoietic lineages markers, CD45, and nuclear images of the cells with 7-aminoactinomycin D (7-AAD). The smaller cells (<6 μm) exhibited higher expression of Sca-1 (Sca-1^{bright}) relative to the larger cells (>6 μm; Sca-1^{dimneg}).

Table 3 summarizes the morphological features of both fractions of Sca-1⁺/Lin^{neg}/CD45^{neg} cells, including size and nuclear to cytoplasmic (N/C) ratio analyzed by the ISS. Sca-1^{bright} cells (<6 μm) were smaller in size and possessed a higher N/C ratio when compared to the Sca-1^{dim} larger cells. The Sca-1^{bright} cells made up 17.35±3.04% of the total Sca-1⁺/Lin^{neg}/CD45^{neg} population (see Table 3). The average size of these cells was 4.88±1.08 μm, and the N/C ratio was 3.19±1.16. The values presented in Table 3 represent average numbers obtained from three independent experiments (Mean±SEM). Fetal livers from 15-20 fetuses were combined in each experiment. Morphometric analysis was performed on at least 100 images of cells from each subpopulation.

FL cells were also fixed and stained for markers of pluripotent stem cells including Oct-4 and SSEA-1, and also for hematopoietic lineages markers (Lin), CD45, and Sca-1. Nuclei were stained with 7-aminoactinomycin D (7-AAD). Magnified nuclear images combined with image of indicated pluripotent markers showed intranuclear expression of Oct-4 and surface appearance of SSEA-1. The majority of cells with the VSEL phenotype and detectable expression of pluripotent markers belonged to the compartment of small (<6 μm) Sca-1⁺/Lin^{neg}/CD45^{neg} cells.

The fraction of smaller FL-derived Sca-1⁺/Lin^{neg}/CD45^{neg} VSELs (i.e., those smaller than 6 μm in diameter) contained cells that expressed both Oct-4 and SSEA-1.

TABLE 3
Characteristics of the Smaller Subpopulation of FL-derived
Sca-1⁺/Lin^{neg}/CD45^{neg} VSELs

| Sca-1⁺/Lin^{neg}/CD45^{neg} Cells | Size (μm) | N/C Ratio |
|---|---|---|
| All Cells in Population | 7.19 ± 0.10 | 2.63 ± 0.48 |
| Cells Smaller than 6 μm | 4.88 ± 1.08 | 3.19 ± 1.16 |
| Cells Larger than 6 μm | 7.75 ± 0.98 | 2.65 ± 0.30 |

### Example 8

### Content of Sca-1⁺/Lin^{neg}/CD45^{neg} and Oct-4/Sca-1⁺/Lin^{neg}/CD45^{neg} VSELs in Fetal and Adult Liver

Based on flow cytometric and ISS analyses, the total number of Sca-1⁺/Lin^{neg}/CD45^{neg} and small Oct-4⁺/Sca-1⁺/Lin^{neg}/CD45^{neg} cells in 12.5, 15.5, and 17.5 dpc FLs an in livers isolated from 4-8 week old adult mice were calculated. The results are presented in Table 4.

| | Organ: | | | |
|---|---|---|---|---|
| | Fetal Liver | | | Adult Liver |
| Age | 12.5 dpc | 15.5 dpc | 17.5 dpc | 4-8 weeks |
| Total Cells | 1.68 ± 0.42 | 14.90 ± 2.90 | 27.05 ± 5.45 | 17.89 ± 6.21 |
| (×10⁶) | | | | |
| Population: | Sca-1⁺/Lin^{neg}/CD45^{neg} | | | |
| Content (%) | 1.33 ± 0.02 | 0.63 ± 0.27* | 0.09 ± 0.03* | 0.12 ± 0.02 |
| Absolute No. | 22.34 ± 5.60 | 93.87 ± 18.30* | 24.35 ± 8.12 | 21.47 ± 4.25 |
| of Cells (×10³) | | | | |
| Absolute No. | 20.96 ± 5.25 | 16.30 ± 3.20 | 5.02 ± 1.67* | 3.79 ± 1.75* |
| of Cells <6 μm | | | | |
| Population: | Oct-4⁺/Sca-1⁺/Lin^{neg}/CD45^{neg} | | | |
| Content (%) | 1.16 ± 0.16 | 0.11 ± 0.04* | 0.03 ± 0.01* | 0.04 ± 0.01 |
| Absolute No. | 19.48 ± 2.75 | 16.54 ± 5.22 | 6.76 ± 1.35* | 6.98 ± 1.38* |
| of Cells (×10³) | | | | |
| Absolute No. | 16.26 ± 2.20 | 12.97 ± 4.77 | 5.00 ± 0.95* | 4.44 ± 0.88* |
| of Cells <6 μm | | | | |

*p < 0.05 vs. 12.5 dpc FL

Table 4 shows changes in the percent content and absolute numbers of Sca-1⁺/Lin^{neg}/CD45^{neg} and small Oct-4⁺/Sca-1⁺/Lin^{neg}/CD45^{neg} VSELs in FL during embryonic development (12.5, 15.5 and 17.5 dpc) as well as in adult murine liver (4-8 weeks). Table 4 shows also the absolute numbers of small cells (<6 μm) which morphologically correspond to VSELs. The absolute numbers were calculated per whole organ and are presented as averages from three independent experiments (Mean±SEM). Fetal livers from 15-20 fetuses were combined in each experiment. Morphometric analysis was performed on at least 100 images of cells from each subpopulation.

The changes in absolute numbers of both cell populations during liver development observed suggested the following. Initially, the FL contained predominantly very small Oct-4⁺/Sca-1⁺/Lin^{neg}/CD45^{neg} cells resembling BM-derived VSELs and some larger Oct-4^{neg}/Sca-1⁺/Lin^{neg}/CD45^{neg} cells with a lower expression of Sca-1 antigen (12.5 dpc). These latter cells appeared to expand rapidly between 12.5 and 15.5 dpc, while the number of Oct-4⁺ VSELs stayed relatively constant. Subsequently, the absolute numbers of both populations decreased between 15.5 and 17.5 dpc, which might be related to their maturation or migration our of the FL and into the BM along with HSCs, as HSCs are known to exit the fetal liver at this stage of embryonic development and migrate to the developing BM microenvironment. Interestingly, the absolute numbers of both Sca-1⁺/Lin^{neg}/CD45^{neg} cells, Oct-4^{neg} VSELs, as well as Oct-4⁺ VSELs residing in the liver at 17.5 dpc was approximately the same as observed in adult (4-8 weeks) organs.

The total number of small Oct-4⁺ VSELs was highest in 12.5 dpc FLs and decreased with maturation. However, the total numbers of small VSELs were similar in 17.5 dpc FLs and livers isolated from adult mice. This rapid decrease in the content of FL-residing VSELs between 15.5 and 17.5 dpc FLs paralleled the decrease in the number of HSCs that leave the FL at about this developmental stage and translocate to the BM microenvironment, where they establish adult hematopoiesis. This is consistent with the FL being a crossroad and expansion site for migrating stem cells, and supports the possibility of FL being a source for BM-residing VSELs.

### Discussion of Examples 5-8

VSELs are characterized by several features of PSCs, such as markers characteristic for embryonic stem cells, open type chromatin in nuclei, the ability to form fetal alkaline phosphatase-positive spheres that comprise primitive cells able to differentiate into all three major lineages when co-cultured with C2C12 cells (see Kucia et al. (2006) Leukemia 20:857-869; Zuba-Surma et al. (2008) Cytometry A 73A:1116-1127; Zuba-Surma et al. (2008) J Cell Mol Med 12:292-303). However, despite the fact that VSELs express Oct-4, Nanog, and Klf-4, they are generally a population of quiescent cells. They proliferate in co-cultures with other cell types (e.g., C2C12 myoblasts), they do not form teratomas in vivo, and they do not complement blastocyst development.

During mouse embryogenesis, the liver develops as an endodermal invagination from the ventral foregut endoderm about 7.5-8.5 dpc (Houssaint (1980) Cell Differ 9:269-279; Jung et al. (1999) Science 284:1998-2003; Rossi et al. (2001) Genes Dev 15:1998-2009; Zaret (2001) Curr Opin Genet Dev 11:568-574; Zaret (2002) Nat Rev Genet 3:499-512). Early in development the FL is the major hematopoietic organ that becomes colonized by yolk sac-derived HSCs at about 9-10 dpc (Zaret (2000) Mech Dev 92:83-88).

The FL also becomes an important site for expansion and differentiation of HSCs during the second trimester of gestation (Zaret (2000) Mech Dev 92:83-88). Eventually, hematopoiesis is shifted out from the liver and into the bone marrow (Tavian & Peault (2005) Int J Dev Biol 49:243-250; Tada et al. (2006) Anat Histol Embryol 35:235-240). CXCR4⁺ HSCs respond to increasing concentration of SDF-1 in developing BM, and translocate to the BM during the third trimester of gestation.

Disclosed herein are experiments that employ flow cytometry and ISS analyses that evaluated whether FL contains a population of cells resembling adult BM-derived VSELs during various time of gestation. It was determined that murine FL contains small Oct-4⁺/Sca-1⁺/Lin^{neg}/CD45^{neg} cells. These cells, expressed SSEA-4 and were able to grow characteristic spheres in co-cultures with C2C12 myoblasts.

The number of FL-derived VSELs was highest in 12.5 dpc FL and subsequently decreased. The decrease in number of VSELs in FL was reminiscent of the decrease in the number of HSCs in this organ at these same developmental stages. Since VSELs express CXCR4 and respond by chemotaxis to SDF-1 gradients, it is likely that they leave this organ together with HSCs and re-locate in the developing BM. A small percentage of these cells, however, stay in the developing liver and are detectable in adult animals.

As such, disclosed herein for the first time is the discovery that a population of VSELs was present in murine FL. These FL-derived VSELs were very small in size, expressed several genes characteristic of PSCs (e.g., Oct-4, Nanog, Rex-1, Dppa3, and Rif1), and in co-cultures with C2C12 cells grew spheres that resembled embryoid bodies. The age-related decrease in their numbers in FL appeared to correlate with the observed decline in the expression of pluripotent genes and formation of VSEL-DS by these cells. From this, it appears likely that VSELs are deposited in developing organs as pools of epiblast-migrating PSCs, some of which translocate along with HSCs to the developing BM.

Disclosed herein are also new strategies that can be used to characterize very small, embryonic-like (VSEL) stem cells (SCs) regarding both their clonality and self-renewal. Strong evidence is provided that VSELs, which do not posses immediate hematopoietic activity (i.e., do not grow colonies in vitro, do not show long term culture initiating-cell (LTCiC) activity in co-cultures over normal stromal cells, do not show spleen colony forming unit (CFU-S) potential, and do not radioprotect lethally irradiated mice), became hematopoietic after expansion on C2C12 or OP9 cells. It is disclosed that in contrast to hematopoietic Sca-1⁺/lin^{neg}/CD45⁺ cells, VSELs that are double-sorted from the same bone marrow (BM) samples as a population of Sca1⁺/lin^{neg}/CD45^{neg} cells did not reveal hematopoietic activity in any of the previously mentioned assays in vitro or in vivo. These results provided evidence that a unique population of cells that is not “contaminated” by hematopoietic Sca-1⁺/lin^{neg}/CD45⁺ cells was isolated.

Also disclosed herein is that Sca-1⁺/lin^{neg}/CD45^{neg} cells isolated from BM were still heterogenous, and that only a subset of these cells were able to acquire hematopoietic potential after co-culture over OP9 or C2C12 cell lines.

Because about 60% of VSELs are SSEA-1⁺ and about 25% are aldehyde dehydrogenase high (ALDH^{hi}), these subpopulations of cells can be sorted and tested for hematopoietic potential to evaluate hematopoietic differentiation of VSELs. Once established, a more highly purified subpopulation of VSELs with hematopoietic potential is acquired and studies at the clonal level are performed

Additionally, a quantitative approach in which a number of VSELs isolated from different organs is disclosed. The ability of these cells to differentiate along the hematopoietic lineage in in vitro co-cultures is studied. In addition, in vivo experiments to address in vivo hematopoietic properties of VSELs are disclosed. In particular, the ability of these cells to home to the bones after intravenous vs. intrabone injection is tested. Also, VSELs are co-transplanted with short-term repopulating hematopoietic SCs (ST-HSCs).

### Example 9

### VSELs to Reverse Anemia in a W/W^{V} Mouse Model

Since lethal irradiation could affect hematopoietic environment and expansion of VSELs, whether VSELs can re-establish normal hematopoiesis is tested by employing a reversal of the W/W^{v} mice macrocytic anemia model (Wiktor-Jedrzejczak et al. (1979) Experientia 35:546-547). This model allows for study of the hematopoietic contribution of transplanted VSELs without conditioning animals for transplantation by irradiation.

Accordingly, W/W^{v} mice (10 per group) are transplanted with VSELs (10-10³/animal) isolated from WT littermates and as control from W/W^{v} mice. Six months after transplantation, whether macrocytic anemia is reversed in these animals is evaluated. It is expected that VSELs from WT mice should have an advantage over VSELs from W/W^{v} mice. If VSELs contribute to hematopoiesis, they should reverse macrocytic anemia in these animals.

### Example 10

### Transplantation into Rag2^{neg/neg}/gc^{neg/neg} Mice

Rag2^{neg/neg}/gc^{neg/neg} female mice (B6 background) are employed as recipients of VSEL-derived hematopoietic cells. Mice (6/group) are irradiated in two doses 4 hours apart by 400 cGy γ-irradiation injected via tail vein with 2×10⁶ B6 GFP⁺ CD45⁺ VSEL-derived OP9-activated HSCs in 400 ml of DMEM/1% FCS. Subsequently, mice are bled every month to evaluate the number of GFP⁺ hematopoietic cells circulating in PB. CFU-S assay: Rag2^{neg/neg}/gc^{neg/neg} female mice (B6 background) are employed as recipients of VSEL-derived OP9-activated hematopoietic cells. Recipient animals are irradiated with 900 cGy γ-irradiation and 10⁵ whole BM or 10⁶ VSEL-derived CD45⁺ hematopoietic cells are injected retroorbitally in 200 ml of PBS. Mice (12/group+6 animals for irradiation control to exclude endogenous CFU-S formation) are sacrificed 12 days after injection of cells. Their spleens are fixed in Bouin's buffer and scored for CFU-S number. These experiments provide additional evidence as to whether cells isolated from VSEL-derived cells activated over OP9 cell cultures can contribute to hematopoiesis in vivo.

### Example 11

### Transplants into Secondary Recipients

Six weeks after transplantation, BM cells are isolated from mice transplanted with GFP⁺ VSELs. BM-derived GFP⁺ cells are sorted by FACS and used to rescue lethally-irradiated WT syngeneic animals. Chimerism in secondary transplanted mice is evaluated as described above.

### REFERENCES

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Basiji et al. (2007) Clin Lab Med 27:653-670.
Crosby et al. (2001) Gastroenterology 120:534-544.
Fiegel et al. (2003) Hepatology 37:148-154.
Houssaint (1980) Cell Differ 9:269-279.
Jung et al. (1999) Science 284:1998-2003.
Krupnick et al. (2004) Tissue Eng 10:723-735.
Kucia et al. (2006) Leukemia 20:857-869.
Kucia et al. (2007) Leukemia 21:297-303.
Lemmer et al. (1998) J Hepatol 29:450-454.
Minguet et al. (2003) J Clin Invest 112:1152-1163.
Nava et al. (2005) Differentiation 73:249-260.
Nierhoff et al. (2005) Hepatology 42:130-139.
Nowak et al. (2005) Gut 54:972-979.
PCT International Patent Application Publication Nos. WO 2007/067280 and 2009/059032.
(1998) Hepatology 27:433-445.
Ratajczak et al. (2003) Stem Cells 21:363-371.
Ratajczak et al. (2004) Leukemia 18:29-40.
Ratajczak et al. (2007) Leukemia 21:860-867.
Ratajczak et al. (2008a) Exp Hematol 36:742-751.
Ratajczak et al. (2008b) J Autoimmun 30:151-162.
Ratajczak et al. (2008c) Stem Cell Rev 4:89-99.
Rossi et al. (2001) Genes Dev 15:1998-2009.
Shin et al. (2008) Blood 112:385.
Suzuki & Nakauchi (2002) Semin Cell Dev Biol 13:455-461.
Suzuki et al. (2000) Hepatology 32:1230-1239.
Suzuki et al. (2002) J Cell Biol 156:173-184.
Tada et al. (2006) Anat Histol Embryol 35:235-240.
Tavian & Peault (2005) Int J Dev Biol 49:243-250.
Zaret (2000) Mech Dev 92:83-88.
Zaret (2001) Curr Opin Genet Dev 11:568-574.
Zaret (2002) Nat Rev Genet 3:499-512.
Zuba-Surma et al. (2007a) Folia Histochem Cytobiol 45:279-290.
Zuba-Surma et al. (2007b) Adv Cell Biol 34:361-375.
Zuba-Surma et al. (2008a) Cytometry A 73A:1116-1127.
Zuba-Surma et al. (2008b) J Cell Mol Med 12:292-303.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. An isolated population of stem cells, wherein the isolated population of stem cells comprises substantially purified CD133⁺/GlyA^{neg}/CD45^{neg} cells isolated from cord blood (CB), wherein the stem cells have hematopoietic competency.

2. The isolated population of claim 1, wherein the CD133⁺/GlyA^{neg}/CD45^{neg} cells are ALDH^{high} cells.

3. The isolated population of claim 1, wherein the CD133⁺/GlyA^{neg}/CD45^{neg} cells are ALDH^{low} cells.

4. The isolated population of claim 1, wherein hematopoietic competency is established by co-culturing the CD133⁺/GlyA^{neg}/CD45^{neg} stem cells in the presence of an OP9 feeder layer for a time sufficient to induce hematopoietic competency.

5. The isolated population of claim 1, wherein hematopoietic competency includes the ability to differentiate to one or more of myeloid cells, B cells, and T cells.

6. The isolated population of claim 1, wherein the CD133⁺/GlyA^{neg}/CD45^{neg} stem cells express one or more of CXCR4 and CD34.

7. The isolated population of claim 1, wherein the CD133⁺/GlyA^{neg}/CD45^{neg} stem cells express one or more of c-met, c-kit, and LIF-R.

8. The isolated population of claim 1, wherein the CD133⁺/GlyA^{neg}/CD45^{neg} stem cells express one or more of SSEA-4, Oct-4, Rev-1, and Nanog.
